(19) United States
(12) Patent Application Publication
(10) Pub. No.: US 2023/0384301 A1
(43) Pub. Date: Nov. 30, 2023

(54) PLASMONIC NANOPARTICLE ASSISTED ENZYME-LINKED IMMUNOSORBENT ASSAY IN A FLUIDICS DEVICE

(71) Applicant: NICOYA LIFESCIENCES, INC., Kitchener (CA)

(72) Inventors: Harish KRISHNAKUMAR, Cambridge (CA); Valentina TAIAKINA, Kitchener (CA); Ryan Cameron DENOMME, Kitchener (CA); Juewen LIU, Waterloo (CA); Zhicheng HUANG, Kitchener (CA)

(21) Appl. No.: 18/249,900

(22) PCT Filed: Oct. 22, 2021
(86) PCT No.: PCT/CA2021/051491

§ 371 Date: Apr. 20, 2023

Related U.S. Application Data
(60) Provisional application No. 63/104,006, filed on Oct. 22, 2020.
(60) Provisional application No. 63/228,970, filed on Aug. 3, 2021.

(51) Int. Cl. G01N 33/543; G01N 33/58; B82Y 5/00

(52) U.S. Cl. CPC G01N 33/54333; G01N 33/581; G01N 33/587; B82Y 5/00; G01N 2333/916

(57) ABSTRACT

Methods for plasmonic nanoparticle assisted detection of target analytes are provided including methods of plasmonic nanoparticle assisted enzyme-linked immunosorbent assay (ELISA) in a fluidics device. For example, a digital microfluidics (DMF) system is provided that includes a DMF device (or cartridge) in which the methods of plasmonic nanoparticle assisted ELISA may be performed. The disclosed methods for detecting target analytes include measuring an optically detectable change caused by one or a combination of etching, growth, aggregation, or altered interparticle distance of plasmonic particles in the vicinity of a target analyte-capture biomolecule complex in response to a product or byproduct generated by enzyme-substrate reactions. In the methods, the amount of enzyme-substrate reactions is proportional to the number of target analytes bound to the capture biomolecules.

### CROSS-REFERENCE TO RELATED APPLICATIONS

This application claims the benefit of priority of U.S. provisional patent application No. 66/104,006 filed on Oct. 22, 2020 and No. 63/228,970 filed on Aug. 3, 2021, the disclosure of which is incorporated herein by this reference in its entirety.

### FIELD

The presently disclosed subject matter relates generally to the detection of molecules, such as DNA, proteins, drugs, and the like, and more particularly to methods for signal enhancement and limit of detection (LOD) improvement in plasmonic nanoparticle assisted enzyme-linked immunosorbent assay (ELISA) in a fluidics device.

### BACKGROUND

In the field of microfluidics, there is difficulty in producing low-cost, sensitive, and portable optical detection systems, especially with respect to portable point-of-care (POC) diagnostics. For example, polymerase chain reaction (PCR) is standard for viral detection, but it requires dedicated equipment and trained lab personnel, making it unsuitable for POC testing. Traditional POC assays are done using lateral flow immunoassays (LFIAs), but LFIAs have inadequate sensitivity for direct viral detection in saliva and are prone to errors. ELISA is the gold-standard for viral antigen analysis, and commercial kits are available. However, like PCR, lab ELISAs are complex, lengthy, and require specialized equipment and trained operators. In addition, the sensitivity and lower detection limits of conventional fluorescent dyes for optical detection (e.g., 3,3′,5,5′-tetramethylbenzidine (TMB)) is limited.

Enzyme-linked immunosorbent assay (ELISA) based detection systems have been widely used for the detection of various kinds of disease biomarkers. 3,3′,5,5′-tetramethylbenzidine (TMB) is an important substrate that can be oxidized to TMB⁺ (blue) or TMB²⁺ (yellow) for colorimetric immunoassays. It was reported that TMB²⁺ can quantitatively and efficiently etch AuNRs, which converted the color of TMB²⁺ to the color change of AuNRs to increase the sensitivity of detection. However, the utility of this method is limited by the extreme conditions required for this reaction to occur. Hydrogen peroxide (H₂O₂) is an important molecule in biology. As a byproduct of many enzymatic reactions, H₂O₂ is a popular target of many sensors. A classic H₂O₂ sensor is based on the color change of 3,3′,5,5′-tetramethylbenzidine (TMB). In the presence of peroxidases (e.g., horseradish peroxidase HRP) and H₂O₂, the chromogenic TMB substrate can be oxidized to colored products TMB⁺. However, the sensitivities of these colorimetric detectors are limited by the final oxidized product concentrations.

Accordingly, new approaches are needed for addressing these limitations and for improving optical detection capabilities in microfluidics.

### SUMMARY

It is an object of the present disclosure to detect and quantify proteins (e.g., antibodies) in a sample using plasmonic nanoparticle assisted ELISA.

Plasmonic nanoparticles described herein include nanoparticles of any type, shape, size, and material composition; although, preferably, one or more of the particle dimensions are less than about 100 nm. For nanoparticles consisting of more than one material composition (e.g., core-shell particles) at least one of the particle layers may consist of a plasmonic layer. Aside from the plasmonic layer, the nanoparticles may also include a dielectric, semiconductor, or polymeric material, and/or any other type of material. In plasmonic nanoparticles having a semiconductor layer, the semiconductor layer may be magnetic or superparamagnetic. Nanoparticle types that may be used with this disclosure include (but are not limited to) core-shell particles, rattle-type particles, hollow particles, porous particles, bimetallic particles, and particles consisting of a single metal composition.

It is another object of the present disclosure to quantify protein concentration in samples through colorimetric detection.

Colorimetric change may be observed through the naked human eye and/or with instrumentation. Colorimetric change or color change described herein, includes (but is not limited to) a change in the intensity of a color and/or perceivable color hues.

In one embodiment, the selection of nanoparticles to be integrated in the present inventive device may be optimized for colorimetric detection by the naked human eye and/or with instrumentation (e.g., smartphone camera). Preferably, the initial and/or final readout color from the device has a maximum fluorescence, absorbance, scattering, and or extinction peak between about 380 nm to about 800 nm for colorimetric detection by the naked human eye. The initial and/or final readout color from the present inventive device may have a maximum fluorescence, absorbance, scattering, and or extinction peak in the ultraviolet, visible, and/or infrared wavelengths for colorimetric detection with instrumentation.

Various methods and designs for plasmonic nanoparticle assisted ELISA in a fluidics device are described herein. The present disclosure describes embodiments of the present disclosure whereby methods for signal and/or limit of detection (LOD) enhancement may be applied to the various methods as further described herein. Some non-limiting examples of colorimetric change for protein detection include etching, aggregation, and/or further growth of plasmonic nanoparticles. Colorimetric change may also be observed due to nucleation and growth of plasmonic nanoparticles by reducing metal ion precursors. Colorimetric change may also be observed due to quenching and/or unquenching the fluorescence of fluorescent probes. The various embodiments described herein generally relate to the use of plasmonic nanoparticles to amplify the signal as a result of substrate conversion in ELISA. One embodiment of the present disclosure relates to signal enhancement and/or LOD improvement in plasmonic nanoparticle assisted ELISA and is not limited to any particular type of color change modality (e.g., nanoparticle etching or aggregation). Other embodiments described herein relate to signal enhancement and/or LOD improvement in etching-based plasmonic nanoparticle assisted ELISA. Yet another embodiment relates to signal enhancement and/or LOD improvement in aggregation-based plasmonic nanoparticle assisted ELISA. Other embodiments described herein involve the use of TMB substrate solution, HRP, and gold nanoparticles (e.g., urchin-like gold nanoparticles (AuNUs), also called gold nanourchins) for plasmonic nanoparticle assisted ELISA. HRP may oxidize TMB to TMB+ or TMB2+ which in turn may etch and/or aggregate gold nanoparticles. In such embodiments, a negative control may be used in the assay. The negative control may consist of gold nanoparticles and TMB substrate solution. The TMB need not be oxidized in the negative control solution since HRP may not be present. Positive samples may contain oxidized TMB due to the presence of HRP.

It is another object of the present disclosure to use a black background to maximize observation of scattered light from plasmonic nanoparticles. For example, a black PCB may be used to provide the black background color to maximize observation of scattered light from the plasmonic nanoparticles. The perceived color of solution containing plasmonic nanoparticles may be different on a black background compared to backgrounds of other colors (such as a white background).

In an etching-based plasmonic nanoparticle assisted ELISA assay, an absorber (e.g., dye) may be added to the control(s) and sample(s) to tune the intensity and/or brightness of the perceived color in the control(s) and sample(s) in the event of a positive sample. As a non-limiting example, plasmonic nanoparticles with high scattering efficiency and a LSPR wavelength near the max absorbance wavelength of the selected absorber may be selected for use in an etching-based plasmonic nanoparticle assisted ELISA assay. As another non-limiting example, an etching-based assay involving gold nanourchins with a LSPR near about 650 nm and methylene blue as the selected absorber may be used. As the gold nanourchins are etched (as a result of enzyme-substrate reactions), the LSPR blue-shifts. As the gold nanourchins are etched, the absorbance and/or reflectance profile of the gold nanourchins may not overlap as much with the absorbance profile of methylene blue. This may result in a perceivable color change but also a change in the intensity of the perceived color due to the light scattered by the gold nanourchins. In this specific non-limiting example, a positive sample (where gold nanourchins are etched) may be of a different color and look brighter (and/or more intense) than the negative control (where gold nanourchins remain unetched).

In an etching-based plasmonic nanoparticle assisted ELISA assay, one or more types of surfactants (e.g., CTAB) may be used to promote or catalyze etching of the plasmonic nanoparticles in the event of a positive sample. The rate and/or extent of etching in the positive samples may be significantly greater than in the negative control. One or more of the surfactant types may be a quaternary ammonium surfactant (e.g., CTAB or CTAC). Aside from promoting or catalyzing plasmonic nanoparticle etching in the positive sample, the surfactant(s) may have additional functionalities as well, including, but not limited to stabilizing the negative control (e.g., preventing or reducing aggregation of plasmonic nanoparticles). The surfactant(s) may also prevent or reduce aggregation of plasmonic nanoparticles in the sample as well (positive or negative). The surfactant(s) may be dispersed in the same solution as the plasmonic nanoparticles or be coated, physically adsorbed, or chemically conjugated onto the plasmonic nanoparticles. In such embodiments, CTAB may be used to stabilize plasmonic nanoparticles (e.g., gold nanourchins) in the substrate solution (e.g., prevent nanoparticle aggregation) and to promote etching of plasmonic nanoparticles in solution in which the substrate has been converted by reporter enzymes. CTAB may prevent aggregation of plasmonic nanoparticles in substrate solution (thereby providing a stable negative control) and promote or catalyze etching of plasmonic nanoparticles after substrate conversion (e.g., in a positive sample).

In an etching-based plasmonic nanoparticle assisted ELISA assay, one or more types of halide ions (e.g., chloride, bromide, and iodide) may be added to solution to promote or accelerate etching of plasmonic nanoparticles in the event of a positive sample. The rate of etching in the positive samples may be significantly greater than in the negative control. Halide ions may be added to a solution in which plasmonic nanoparticles are present in the form of a cationic surfactant, as a salt, or in another form. Halide ions may form complexes with byproducts of enzyme-substrate reactions (in a positive sample) that may etch plasmonic nanoparticles faster, more effectively, or in a different manner (e.g., selectively etch specific facets) compared to solely using halide ions or the byproducts of the enzyme-substrate reactions.

In an etching-based plasmonic nanoparticle assisted ELISA assay, one or more types of thiosulfates (e.g., ammonium thiosulfate or sodium thiosulfate) and/or thiosulfonates may be added to solution to promote or accelerate etching of plasmonic nanoparticles in the event of a positive sample. The rate and/or extent of etching in the positive sample may be significantly greater than in the negative control. Thiosulfates and/or thiosulfonates may form complexes with byproducts of enzyme-substrate reactions (in a positive sample) that may etch plasmonic nanoparticles faster, more effectively, or in a different manner (e.g., selectively etch specific facets of the nanoparticles) compared to solely using thiosulfates/thiosulfonates or the byproducts of the enzyme-substrate reactions. As a non-limiting example, sodium thiosulfate may etch gold nanorods slowly in dissolved oxygen but much faster in the presence of oxidized TMB (TMB+ and/or TMB2+).

In an etching-based plasmonic nanoparticle assisted ELISA assay, one or more types of metal ions (e.g., Cu2+ or Fe3+) may be added to solution to promote or accelerate etching of plasmonic nanoparticles in the event of a positive sample. The rate of etching in the positive samples may be significantly greater than in the negative control. Metal ions may provide a synergetic effect to other etchants produced as byproducts of enzyme-substrate reactions (in a positive sample) thereby etching plasmonic nanoparticles faster, more effectively, or in a different manner (e.g., selectively etch specific facets of the nanoparticles) compared to solely using metal ions or the byproducts of the enzyme-substrate reactions.

It is yet another object of the present disclosure to use a combination of surfactants, halide ions, thiosulfates/thiosulfonates, and metal ions to promote or accelerate etching of plasmonic nanoparticles in the event of a positive sample. The rate of etching in the positive samples may be significantly greater than in the negative control.

In an aggregation-based plasmonic nanoparticle assisted ELISA assay, one or more types of polymers may be added to solution to promote or accelerate aggregation, flocculation, and/or reduce interparticle distance of plasmonic nanoparticles in the event of a positive sample. Polymers may be ionic or non-ionic. Polymers may promote or accelerate nanoparticle aggregation and/or flocculation through one or more mechanisms, including but not limited to the mechanisms described below. Polymers may provide a synergistic effect to other aggregants, flocculants, and/or coagulants produced as byproducts of enzyme-substrate reactions (in a positive sample) to reduce interparticle distance between plasmonic nanoparticles and thereby providing a greater colorimetric signal enhancement and/or LOD improvement. A non-ionic polymer (e.g., TWEEN 20) may provide a synergistic effect to oxidized TMB (which is converted from TMB substrate by reporter enzymes such as HRP) in aggregating plasmonic nanoparticles (e.g., citrate coated gold nanourchins). A non-ionic polymer (e.g., Polysorbate 20) may flocculate plasmonic nanoparticles through polymer bridging in tandem with charge neutralization of the plasmonic nanoparticles byproducts in enzyme-substrate reactions in positive samples. One or more of the byproducts produced in the enzyme-substrate reactions may be of an opposite charge to the initial surface charge of the plasmonic nanoparticles to induce charge neutralization.

In one embodiment, a plasmonic particle assisted method of detecting a target analyte, is provided, the method including:
a. introducing a sample fluid potentially comprising a target analyte to a capture biomolecule in a microfluidic device, wherein binding of the target analyte to the capture biomolecule forms a target-capture biomolecule complex, and wherein, optionally, one or both of the capture biomolecule and a plasmonic particle is immobilized on a surface;
b. introducing an antibody that binds directly or indirectly to the target analyte at a different site than the capture biomolecule, wherein:
   (i) an enzyme is conjugated directly or indirectly to the antibody, or
   (ii) the enzyme is conjugated to the capture biomolecule;
c. introducing a substrate for the enzyme;
d. introducing a plasmonic particle;
e. measuring an optically detectable change caused by one or a combination of etching, growth, aggregation, or altered interparticle distance of the plasmonic particle in the vicinity of the target-capture biomolecule complex, wherein the etching, growth, aggregation, or altered interparticle distance is in response to a product or byproduct generated by a reaction between the enzyme and the substrate, wherein the amount of enzyme-substrate reactions is proportional to the number of target analytes bound to the capture biomolecules; and
f. optionally, quantifying an amount of the target analyte present in the sample based on the optically detectable change.

In the method, the surface can be a magnetic bead. The capture biomolecule can be immobilized on the surface or on the magnetic bead and the plasmonic particle can be introduced in a fluidic suspension subsequent to introduction of the substrate.

In the method, the plasmonic particles can be introduced as a fluidic suspension in a droplet in conjunction with the substrate or the plasmonic particles can be introduced as a fluidic suspension in a droplet separately from the substrate.

In one embodiment, the capture biomolecule and the plasmonic particle are both immobilized on the surface. In another embodiment, the capture biomolecule is immobilized on the surface and the plasmonic particle is immobilized on a separate surface.

In the method, the measuring of the optically detectable change can be performed by one or both the naked eye, with an instrument, or with a camera.

In the method, the sample fluid can be a bodily fluid from a human or an animal. The target analyte can be a protein, an antigen, an antibody, an IgG antibody, an IgM antibody, a virus, a molecule or molecular structure from a virus, a bacteria, or any other pathogen, or a molecule or molecular structure bound to the outer surface of a virus, a bacteria, or any other pathogen. An internal target molecule or target molecular structure can be exposed by disrupting the integrity of the virus, the bacteria, or any other pathogen prior to introducing the sample to the microfluidic device.

In one embodiment of the method, the target analyte is a target antibody and the capture biomolecule is an antigen that the target antibody binds to. In another embodiment, the target analyte is a virus and the capture biomolecule is an antibody that binds to the virus.

In the method, the optically detectable change can be a colorimetric change.

In one embodiment of the method, the capture biomolecule is immobilized on the surface, the surface is a magnetic bead, and the antibody is conjugated indirectly to the enzyme as a result of both the antibody and the enzyme being conjugated to a second surface that can be, for example, a non-magnetic bead.

In another embodiment, the capture molecule is immobilized on the surface which is a magnetic bead, and the antibody is immobilized on a second surface.

In one embodiment of the method, the enzyme is horseradish peroxidase (HRP), the substrate is TMB, and the optically detectable change is a result of etching of the plasmonic particles by the oxidized TMB substrate.

In another embodiment, the enzyme is alkaline phosphatase (AP).

In the method, one or more dimensions of the plasmonic particles is less than about 100 nm. In various embodiments, the plasmonic particles have more than one layer, more than one material, or combinations thereof. The plasmonic particles can have one or a combination of a plasmonic layer, a dielectric layer, a semiconductor layer, or a polymeric layer. The semiconductor layer can be a magnetic, a paramagnetic, or a superparamagnetic semiconductor layer. The plasmonic particle can be a core-shell particle, a rattle-type particle, a hollow particle, a porous particle, a bimetallic particle, a single metal particle, a gold nanorod, or a gold nanourchin. The core-shell particle can include a gold core and an outer porous (or mesoporous) metal oxide layer, thereby facilitating enzyme catalyzed etching of the particle core for optical detection of the target analyte. The core-shell particle can include a silver core and an outer porous (or mesoporous) metal oxide layer, thereby facilitating enzyme catalyzed etching of the particle core for optical detection of the target analyte. The metal oxide layer can be silicon dioxide (i.e., SiO2@Ag or SiO2@Au).

In one embodiment, the method further includes introducing a fluorescent probe. The fluorescent probe can be adhered or chemically bound to the plasmonic particle. The fluorescent probe can be a quantum dot. The optically detectable change can be caused by etching of the plasmonic particles which allows the fluorescent probe or quantum dot to be released or detached from the plasmonic particles. In the method including introducing a fluorescent probe, the method can further include introducing a fluorescence quencher/acceptor moiety. The fluorescence quencher/acceptor moiety can be a black hole quencher. The fluorescence quencher/acceptor moiety can be a short oligomer dual-labeled with a FRET pair. The short oligomer can be a peptide, an aptamer, or a carbohydrate-based molecule.

In the method, the plasmonic particle can include two or more types of plasmonic particles, thereby increasing the sensitivity and/or the range of detection for the target analyte. The plasmonic particles can be a mixture of one or more aggregation states of the same type of plasmonic particle, thereby increasing the sensitivity and/or range of detection for the target analyte. The plasmonic particles can be a mixture of one or more aggregation states of one or more different types of plasmonic particles, thereby increasing the sensitivity and/or range of detection for the target analyte.

In the method, the optically detectable change can be a colorimetric change, and the method can further include using a background color that is not changed in response to the enzyme-substrate reactions, thereby providing a constant measurable color. The background color can be provided by a dye. The background color be provided by a second plasmonic particle that is not changed in response to the enzyme-substrate reactions. The second plasmonic particle can have a protective surface modification, thereby providing resistance to a colorimetric change in response to the enzyme-substrate reactions.

In the method, the reduced interparticle distance can be mediated by an addition or a presence of ionic molecules containing one or more functional groups that can covalently bind to the particles, where the addition or the presence of the ionic molecules is a result of the enzyme-substrate reactions. The one or more functional groups can include one or a combination of a carboxyl group, a thiol group, or an amine group. In one example, the one or more functional groups are present on the plasmonic particles, and in response to the enzyme-substrate reactions a chemical bond is formed between the plasmonic particles, thereby aggregating or reducing the interparticle distance of the plasmonic particles. The chemical bonding of the plasmonic particles can be provided by one or more different functional groups used in a click chemistry reaction. The click chemistry reaction can be a thiol-ene reaction or a copper(I)-catalyzed azide-alkyne cycloaddition.

In one embodiment of the method, the plasmonic particle is a core-shell particle and the method further includes introducing a metal ion precursor. The optically detectable change is caused by growth of the plasmonic particle in response to reduction of the metal ion precursor mediated by the enzyme-substrate reactions. In this case, the core-shell particle includes a thin shell formed in response to the enzyme-substrate reactions on top of a plasmonic core. The plasmonic core and the thin shell can be the same metallic material.

In one embodiment, a method is provided for detecting a target analyte, where the method includes:
a. introducing a sample fluid potentially comprising a target analyte to a capture biomolecule in a microfluidic device, wherein binding of the target analyte to the capture biomolecule forms a target-capture biomolecule complex, and wherein, optionally, one or both of the capture biomolecule and a plasmonic particle is immobilized on a surface;
b. introducing an antibody that binds directly or indirectly to the target analyte at a different site than the capture biomolecule, wherein:
   (i) an enzyme is conjugated directly or indirectly to the antibody, or
   (ii) the enzyme is conjugated to the capture biomolecule;
c. introducing a substrate for the enzyme and a metal ion precursor, wherein the introducing is either at the same time or at separate times;
d. measuring an optically detectable change caused by nucleation and growth of a plasmonic particle in the vicinity of the target-capture biomolecule complex, wherein the optically detectable change is in response to a reduction in the metal ion precursor as a result of the nucleation and growth of the plasmonic particle mediated by a reaction between the enzyme and the substrate, wherein the amount of enzyme-substrate reactions is proportional to the number of target analytes bound to the capture biomolecules; and
e. optionally, quantifying an amount of the target analyte present in the sample based on the optically detectable change.

In another embodiment, a system is provided for performing plasmonic particle assisted detection of a target analyte, the system including:
a. a digital microfluidic (DMF) cartridge configured for plasmonic particle assisted enzyme-linked immunosorbent assay (ELISA) as described in the present disclosure, the DMF cartridge comprising a plurality of plasmonic nanoparticles for performance of the ELISA;
b. an illumination source arranged in proximity to the digital microfluidic cartridge for providing light;
c. an optical measurement device arranged in proximity to the digital microfluidic cartridge for obtaining optically detectable readings including one or a combination of light intensity, color, and hue; and
d. optionally, a thermal control mechanism for controlling the operating temperature of the digital microfluidic cartridge.

### BRIEF DESCRIPTION OF DRAWINGS

Having thus described the presently disclosed subject matter in general terms, reference will now be made to the accompanying drawings, which are not necessarily drawn to scale, and wherein:

FIG. 1 illustrates a block diagram of an example of a DMF system that includes a DMF cartridge for performing the presently disclosed methods of plasmonic nanoparticle assisted ELISA;

FIG. 2 shows a table indicating an example of color change modalities as plasmonic nanoparticles are etched in ELISA;

FIG. 3 shows a table indicating an example of properties of spherical Ag@Au core-shell particles and gold nanorods;

FIG. 4 shows a table indicating an example of colorimetric plasmonic ELISA based on etching a dual nanoparticle system;

FIG. 5 illustrates a schematic diagram of an example of an etching-based plasmonic ELISA process in a fluidics device in which colorimetric NPs are in suspension;

FIG. 6 illustrates a schematic diagram of an example of an etching-based plasmonic ELISA process in a fluidics device in which colorimetric NPs are immobilized on a surface;

FIG. 7 illustrates a schematic diagram of an example of an etching-based plasmonic ELISA process in a fluidics device in which colorimetric NPs are immobilized on a surface and antigens are bound to the plasmonic NPs;

FIG. 8A, FIG. 8B, FIG. 8C, and FIG. 8D show a table indicating some examples of etching plasmonic nanoparticles;

FIG. 9 illustrates a schematic diagram of an example of an aggregation-based plasmonic ELISA process in a fluidics device;

FIG. 10A illustrates a schematic diagram of an example of a FRET-based plasmonic ELISA process in a fluidics device;

FIG. 10B is a schematic diagram of an example of a process of colorimetric change by enzyme catalyzed cascade in which the activity of a first enzyme in the presence of the target analyte activates a second “reporting” enzyme that produces a product that etches a plasmonic nanoparticle;

FIG. 11 illustrates a schematic diagram of an example of a process of colorimetric detection of multiple proteins in a fluidics device using multimodality plasmonic nanoparticle sensors;

FIG. 12A illustrates a schematic diagram of an example of a process of signal amplification in a fluidics device by binding multiple multi-functionalized magnetic nanoparticles to target viral particles;

FIG. 12B illustrates a schematic diagram of an example of an etching-based plasmonic ELISA process in a fluidics device where the target is a viral particle, the capture biomolecule is attached to a magnetic nanoparticle (MNP) and the enzyme is conjugated to the antibody;

FIG. 12C illustrates a schematic diagram of an example of an etching-based plasmonic ELISA process in a fluidics device where the target is a viral particle, the capture biomolecule is attached to a magnetic nanoparticle (MNP) and the antibody and the enzyme are attached to a non-magnetic bead;

FIG. 12D illustrates a schematic diagram of an example of an etching-based plasmonic ELISA process in a fluidics device where the target is a viral particle, the capture biomolecule and enzyme are attached to a magnetic nanoparticle (MNP);

FIG. 13 illustrates a schematic diagram of an example of a process of colorimetric signal amplification in a fluidics device by binding a single target protein to multi-functionalized magnetic nanoparticles containing multiple reporter enzymes;

FIG. 14 is a schematic diagram of an example of a process 1400 of colorimetric change by etching gold nanoparticles;

FIG. 15 is a schematic diagram of an example of a process 1500 of colorimetric change by color accumulation or depletion;

FIG. 16 is a schematic diagram of an example of a process 1600 of colorimetric change by enzyme catalyzed cascade;

FIG. 17 is a schematic diagram of an example of a process 1700 of colorimetric change by HRP on a non-magnetic nanoparticle;

FIG. 18 is a schematic diagram of an example of a process 1800 of colorimetric change using a particle or bead that has a magnetic core and gold coating or shell;

FIG. 19 is a schematic diagram of another example of a process 1900 of colorimetric change using a particle or bead that has a magnetic core and gold coating or shell;

FIG. 20 is a schematic diagram of an example of a process 2000 of using plasmonic nanoparticle assisted ELISA 112 of DMF system 100 with respect to a CRISPR assay;

FIG. 21 is a schematic diagram of an example of a process 2100 of using plasmonic nanoparticle assisted ELISA 112 of DMF system 100 with respect to another CRISPR assay;

FIG. 22A shows a TEM image of AuNUs-38 NPs before the addition of TMB⁺;

FIG. 22B shows a TEM image of AuNUs-38 NPs after the addition of TMB⁺;

FIG. 22C shows a plot of the UV-vis absorbance spectra of AuNUs-38 NPs before and after etching. Inset photos are the color of Au corresponding samples;

FIG. 22D shows a plot of the distribution histograms of the size change of AuNUs-38 NPs;

FIG. 23A shows a schematic diagram of the etchings of AuNUs and AuNRs by TMB⁺ and TMB²⁺ respectively;

FIG. 23B are grayscale images of the color changes of three AuNPs incubated with increasing TMB⁺ concentration in which the AuNRs in the top row remained a consistent gold color, the AuNUs-38 in the middle row started out a greenish brown color at 0 TMB⁺ on the left and very gradually turned a greenish purple color by 2.75 and 3.76 μM TMB⁺, and the AuNUs-13 on the bottom row started out a bright teal color at 0 TMB⁺, turned a greenish purple color by 0.48 μM TMB⁺ and remained a light purple color from 1.27-3.76 μM TMB⁺;

FIG. 23C shows a plot of SPR peak shifts of AuNRs, AuNUs-38, and AuNUs-13 after one-hour incubation with various TMB⁺ concentrations;

FIG. 23D shows a plot of the absorption spectra measurements of AuNRs reacted with TMB²⁺ in the presence of CTAC;

FIG. 23E shows a plot of the absorption spectra measurements of AuNRs reacted with TMB²⁺ in the presence of CTAB;

FIG. 24A shows a plot of the SPR peak shifts of AuNUs-38 etched in the presence of 0.1% surfactants and wherein 0.1% CTAC & CTAB were respectively 3.1 mm and 2.7 mm;

FIG. 24B shows a plot of the normalized absorption spectra of AuNUs incubated with the mixture of various NaBr concentrations and 4 μm TMB⁺;

FIG. 24C shows a plot of the effects of F⁻ (chloride) on the etching of AuNUs-38 in the presence of 5 mm CTAC and 4 μm TMB⁺;

FIG. 24D shows a plot of the effects of Cl⁻ (fluoride ions) on the etching of AuNUs-38 in the presence of 5 mm CTAC and 4 μm TMB⁺;

FIG. 24E shows a plot of SPR peak shifts of AuNUs-38 NPs as a function of NaBr;

FIG. 24F shows a plot of SPR peak shifts of AuNUs-38 NPs as a function of CTAC;

FIG. 25A shows a plot of the etching kinetics of AuNUs-38 where SPR peak shifts were used;

FIG. 25B shows a plot of the SPR peak shifts of AuNUs-38 which were incubated in different pH environments;

FIG. 25C shows a plot of the stabilities of TMB⁺ produced by UV light at different pHs;

FIG. 25D shows a plot of the zeta-potentials of AuNUs-38 at different pHs;

FIG. 26A shows a TEM image of AuNUs synthesized from 13 nm spherical AuNPs;

FIG. 26B shows a schematic diagram of the etching of AuNUs induced by the product of HRP-catalysed TMB;

FIG. 26C are images in grayscale of the color changes of the AuNPs used in the described method with the increase of H₂O₂ concentration where, the top row wells are a bluish green color at 0 and 200 nM H₂O₂, a purplish color at 400 nM, and a reddish purple at 600 nM and greater concentration of H₂O₂, where the middle row wells are consistently transparent and colorless, and where the bottom row wells are consistently a bluish green color like the 0 and 200 nM H₂O₂ wells of the top row;

FIG. 26D shows a plot of the LSPR shifts of AuNUs-13 as a function of H₂O₂ concentration. Inset: the response at a low concentration range;

FIG. 27 shows a plot of the UV-vis absorption spectra of 13 nm and 38 nm Au seeds;

FIG. 28 shows a plot of the UV-vis absorption spectrum of four times diluted TMB oxidation product in pH 4 acetate buffer;

FIG. 29A shows a TEM image of AuNRs;

FIG. 29B shows a plot of the UV-vis spectra of AuNRs etched by various concentrations of TMB⁺ in the presence of 5 mm CTAB without heating;

FIG. 30 shows a plot of the SPR peak shifts of AuNUs-38 etching as a function of CTAB concentrations;

FIG. 31 shows a plot of the UV-vis spectra of AuNUs-38 NPs incubated with 5 mm NaI for 30 min in which I⁻ ions can etch AuNUs without the addition of TMB⁺;

FIG. 32A shows a plot of the UV-vis spectra of AuNUs-38 etched in the presence of 0.25 mm CTAB 0.75 mm CTAC;

FIG. 32B shows a plot of the UV-vis spectra of AuNUs-38 etched in the presence of 0.5 mm CTAB 0.5 mm CTAC;

FIG. 32C shows a plot of the UV-vis spectra of AuNUs-38 etched in the presence of 0.75 mm CTAB 0.25 mm CTAC in which a greater blue shift happened with higher portion of CTAB (Δλ3>Δλ2>Δλ1);

FIG. 33 shows a plot of the UV-vis spectra of AuNUs-38 in the presence of various NaBr concentrations in whichslight aggregation of AuNUs-38 happened with the addition of 15 mm NaBr;

FIG. 34 shows a plot of the stabilities of AuNUs-13 and AuNUs-38 in different concentrations of H₂O₂;

FIG. 35 shows a plot of the UV-vis spectra change of AuNUs-13 NPs with and without the addition of TMB⁺;

FIG. 36A shows a plot of the etching kinetics of AuNUs-13 NPs where SPR peak shifts were used; and

FIG. 36B shows a plot of the SPR peak shifts of AuNUs-13 NPs incubated in different pH environments in which acetate buffers were used for pH 4 & 5; phosphate buffers were used for pH 6-8, and when Δλ was negative, the aggregation of AuNUs-13 NPs happened

FIG. 37 is a graph showing the maximum LSPR peak position of gold nanourchins mixed with TMB incubated by biotin-HRP conjugated beads blue-shifted significantly more than the NEG.CTL of gold nanourchins that received TMB incubated with streptavidin coated magnetic beads.

FIG. 38 is a graph showing the maximum LSPR peak position of gold nanourchins mixed with TMB incubated by beads that mixed with 250 pM spike protein blue-shifted significantly more than the NEG.CTL of gold nanourchins that received TMB incubated with magnetic beads mixed with buffer instead of 250 pM spike protein.

### ACRONYMS

“AgNPs” is the acronym for “silver nanoparticles.”
“Ag@AuNPs” is the acronym for “silver coated gold nanoparticles.”
“ALP” is the acronym for “alkaline phosphatase.”
“AuNPs” is the acronym for “gold nanoparticles.”
“AuNRs” is the acronym for “gold nanorods.”
“AuNUs” is the acronym for “urchin-like gold nanoparticles.”
“CRISPR” is the acronym for “clustered regularly interspaced short palindromic repeats.”
“CTAB” is the acronym for “cetyltrimethylammonium bromide.”
“CTAC” is the acronym for “cetyltrimethylammonium chloride.”
“DMF” is the acronym for “digital microfluidics.”
“ELISA” is the acronym for “enzyme-linked immunosorbent assay.”
“Fe3O4” is the acronym for “iron oxide.”
“FRET” is the acronym for “Forster resonance energy transfer.”
“HRP” is the acronym for “horseradish peroxidase.”
“IgG” is the acronym for “immunoglobulin G” (i.e., antibody IgG).
“IgM” is the acronym for “immunoglobulin M” (i.e., antibody IgM).
“LOD” is the acronym for “limit of detection.”
“LSPR” is the acronym for “localized surface plasmon resonance.”
“mAb” is the acronym for “monoclonal antibody.”
“MNP” is the acronym for “magnetic nanoparticle” (also referred to herein as a “magnetic bead”).
“NPs” is the acronym for “nanoparticles.”
“OD” is the acronym for “optical density.”
“PCB” is the acronym for “printed circuit board.”
“Pd” is the acronym for “palladium.”
“SiO2” is the acronym for “silica.”
“SiO2@Ag” is the acronym for “silica coated silver nanoparticles.”
“TMB” is the acronym for “3,3′,5,5′-tetramethylbenzidine.”

### General Definitions

“Absorber” means a dye, chromophore, nanoparticle, or other species which absorbs wavelengths of light.

“Byproducts” mean any one or more products or side products produced by an enzyme reaction that is not the primary product.

“Capture biomolecules” means biomolecules (e.g., antigens, antibodies etc.) to which target analytes bind. Thus, in some instances, the terms “capture biomolecule” and “antigen” and “antibody” are used interchangeably herein. For the purposes of the claims, the term “capture biomolecule” refers to the first capture biomolecule (which in some examples is an antibody or an antigen) that is introduced to the target analyte to form a target-capture biomolecule complex. For the purposes of the claims, the term “antibody” refers to an antibody that binds directly or indirectly to the target analyte at a different site or epitope than the capture biomolecule and is introduced after introduction of the capture biomolecule. Thus, the term “target antibody” refers to a target analyte that is an antibody and is not the same as the term “antibody” or the term “detection antibody” or the term “secondary antibody”. The terms “antibody”, “secondary antibody” and “detection antibody” are herein used interchangeably for the purposes of the specification and claims. “Controls” means positive and negative controls.

“Core-shell nanoparticles” means nanoparticles that consist of a core particle encapsulated by a shell.

“Infrared wavelengths” means wavelengths of from about 700 nm to about 1000 nm.

“Localized surface plasmon resonance” means the collective oscillation of electrons at the interface of metallic structures.

“Nanoparticles” means particles with one or more dimensions less than 100 nm.

“Nanourchins” means nanoparticles with a spiky uneven surface.

“Negative control” means a solution containing nanoparticles and other components which should not undergo a colorimetric change.

“Optical density” means a measure of the amount of light that is transmitted through a solution. The solution may or may not contain plasmonic nanoparticles. It is the negative logarithm of the percentage of light transmitted through a solution.

“Oxidized TMB” refers to TMB+ or TMB2+.

“Particles” means particles with one or more dimensions greater than 100 nm.

“Plasmonic nanoparticles” or “plasmonic particles” mean particles whose electron density can couple with electromagnetic radiation of wavelengths larger than the particle. Plasmonic nanoparticles exhibit intense light absorbance, scattering, and/or extinction properties. Plasmonic nanoparticles typically consist of at least one layer or component of noble metals (e.g., gold, silver, palladium, platinum, etc.).

“Positive control” means a solution containing nanoparticles and other components which should undergo a colorimetric change.

“Positive sample” means samples which contain detectable levels of the target analyte.

“Rattle-type nanoparticles” mean nanoparticles that consist of a shell encapsulating one or more freely moving core particles in a solvent.

“Reporter enzymes” mean enzymes that perform a chemical reaction (e.g., substrate conversion) which leads directly or indirectly to a detectable optical change such as, for example, a color change. For the purposes of the specification and claims, the terms “reporter enzyme” and “enzyme” are herein used interchangeably.

“Sample” means fluid that is tested for detection and, optionally, quantification of one or more target analytes. The fluid may be artificially spiked with target analytes, including, for example, but not limited to antibodies, antigens, proteins, viruses, bacteria and other pathogens, and/or other constituents. The fluid may also be collected from humans or animals, such as sweat, saliva, blood, urine, mucous, tear fluid, etc.

“Substrate” means a molecule which gets acted upon by enzymes (e.g., reporter enzymes). In some examples, reporter enzymes catalyze chemical reactions involving the substrate.

“Ultraviolet wavelengths” means wavelengths of from about 10 nm to about 400 nm.

“Visible Wavelengths” means wavelengths of from about 400 nm to about 800 nm.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides methods for signal enhancement and limit of detection (LOD) improvement in plasmonic nanoparticle assisted enzyme-linked immunosorbent assay (ELISA) in a fluidics device.

In some embodiments, a digital microfluidics (DMF) system that includes a DMF cartridge is provided for performing the presently disclosed methods of plasmonic particle assisted ELISA for the detection and quantification of, for example, proteins (e.g., antibodies), viruses, bacteria, and/or any other pathogens.

In some embodiments, using the presently disclosed methods of plasmonic particle assisted ELISA in the DMF system and/or DMF cartridge provides increased sensitivity and lower detection limits as compared with conventional dyes (e.g., 3,3′,5,5′-tetramethylbenzidine (TMB)).

In some embodiments, the presently disclosed methods of plasmonic particle assisted ELISA in the DMF system and/or DMF cartridge may be portable and may be used in, for example, point-of-care (POC) applications.

In some embodiments, using the presently disclosed methods of plasmonic particle assisted ELISA in the DMF system and/or DMF cartridge provides selective detection of multiple proteins (e.g., antibodies IgG and IgM) using, for example, multi-modality plasmonic sensors.

In some embodiments, using the presently disclosed methods of plasmonic particle assisted ELISA in the DMF system and/or DMF cartridge provides detection and quantification of, for example, proteins (e.g., antibodies), viruses, bacteria, and/or any other pathogens. For example, using plasmonic particle assisted ELISA, plasmonic particles of various shapes, sizes, and compositions, which display tunable localized surface plasmon resonance peaks and/or fluorescence quenching through Forster resonance energy transfer (FRET), may be processed (e.g., etched, grown, cleaved and/or aggregated in response to selective detection of proteins (e.g., antibodies)).

In some embodiments, using the presently disclosed methods of plasmonic particle assisted ELISA in the DMF system and/or DMF cartridge, colorimetric change may be observed due to (1) etching of plasmonic particles; (2) aggregation of plasmonic particles; (3) growth of plasmonic particles; (4) nucleation and growth of plasmonic particles by reducing metal ion precursors, and (5) quenching and/or unquenching the fluorescence of fluorescent probes (e.g., quantum dots).

In some embodiments, the presently disclosed methods of plasmonic particle assisted ELISA in the DMF system and/or DMF cartridge may include, but are not limited to, an etching-based plasmonic ELISA process in which colorimetric particles are in suspension, an etching-based plasmonic ELISA process in which colorimetric particles are immobilized, an etching-based plasmonic ELISA process in which colorimetric particles are immobilized and antigens are bound to the plasmonic particles, an aggregation-based plasmonic ELISA process, a FRET-based plasmonic ELISA process, a colorimetric detection of multiple proteins process, a signal amplification process, and a colorimetric signal amplification process.

In some embodiments, the presently disclosed methods of plasmonic particle assisted ELISA in the DMF system and/or DMF cartridge may include enzyme catalyzed cascade reaction ELISA for DMF.

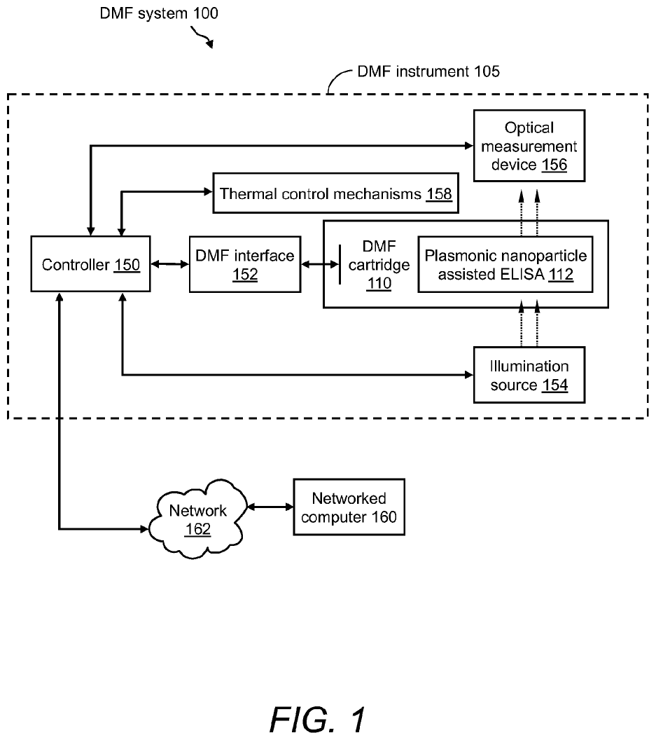

Referring now to FIG. 1 is a block diagram of an example of a DMF system 100 that includes a DMF cartridge 110 for performing the presently disclosed methods of plasmonic particle assisted ELISA for the detection and, optionally, quantification of, for example, proteins (e.g., antibodies), viruses, bacteria, and/or any other pathogens. For example, certain plasmonic particle assisted ELISA 112 may be run on DMF cartridge 110 for the detection and/or quantification of, for example, proteins (e.g., antibodies), viruses, bacteria, and/or any other pathogens. DMF system 100 may be, for example, a plasmon resonance (PR) system or a localized surface plasmon resonance (LSPR) system.

DMF cartridge 110 may facilitate DMF capabilities generally for fluidic actuation including droplet merging, splitting, dispensing, diluting, and the like. One application of these DMF capabilities may be sample preparation. However, the DMF capabilities may be used for other processes, such as waste removal. DMF cartridge 110 of DMF system 100 may be provided, for example, as a disposable and/or reusable cartridge.

DMF system 100 may further include a controller 150, a DMF interface 152, an illumination source 154, an optical measurement device 156, and thermal control mechanisms 158. Controller 150 may be electrically coupled to the various hardware components of DMF system 100, such as to DMF cartridge 110, illumination source 154, and optical measurement device 156. In particular, controller 150 may be electrically coupled to DMF cartridge 110 via DMF interface 152, wherein DMF interface 152 may be, for example, a pluggable interface for connecting mechanically and electrically to DMF cartridge 110. Together, DMF cartridge 110, controller 150, DMF interface 152, illumination source 154, and optical measurement device 156 may comprise a DMF instrument 105.

Controller 150 may, for example, be a general-purpose computer, special purpose computer, personal computer, tablet device, smartphone, smart watch, microprocessor, or other programmable data processing apparatus. Controller 150 may provide processing capabilities, such as storing, interpreting, and/or executing software instructions, as well as controlling the overall operations of DMF system 100. The software instructions may comprise machine readable code stored in non-transitory memory that is accessible by the controller 150 for the execution of the instructions. Controller 150 may be configured and programmed to control data and/or power aspects of these devices. For example, with respect to DMF cartridge 110, controller 150 may control droplet manipulation by activating/deactivating electrodes. Generally, controller 150 may be used for any functions of the DMF system 100. For example, controller 150 may be used to authenticate the DMF cartridge 110 in a fashion similar to how printer manufacturers check for their branded ink cartridges, controller 150 may be used to verify that the DMF cartridge 110 is not expired, controller 150 may be used to confirm the cleanliness of the DMF cartridge 110 by running a certain protocol for that purpose, and so on.

Additionally, in some embodiments, DMF cartridge 110 may include capacitive feedback sensing. For example, a signal may be generated or detected by a capacitive sensor that can detect droplet position, velocity, and size. Further, in other embodiments, instead of capacitive feedback sensing, DMF cartridge 110 may include a camera or other optical device to provide an optical measurement of the droplet position, velocity, and size, which can trigger controller 150 to re-route the droplets at appropriate positions. The feedback may be used to create a closed-loop control system to optimize droplet actuation rate and verify droplet operations are completed successfully.

Further, in some embodiments, controller 150 may be external to DMF instrument 105 (not shown in FIG. 1). The functions described above may be done remotely, for example via a mobile application running on a mobile device connected to various components (i.e. Illumination source 154, among others) via a local network or other network. Output from optical measurement device 156 may also be sent to an external controller 150 via such networks, and displayed on the mobile application or another mobile app running on a mobile device specific for the external controller 150.

Optionally, DMF instrument 105 may be connected to a network. For example, controller 150 may be in communication with a networked computer 160 via a network 162. Networked computer 160 may be, for example, any centralized server or cloud server. The servers may be, for example, virtual servers, with logical drives distributed across geographically diverse physical drives. Network 162 may be, for example, a local area network (LAN) or wide area network (WAN) for connecting to the internet. Though FIG. 1 shows a single Networked computer 160, multiple computers (physical or virtual) may be connected to DMF system 100 via Network 162. For example, one computer may be used to control illumination source 154, another computer may be used to control thermal control mechanism 158, and another computer that is optimized for storing information may be used to store data received from optical measurement device 156, and yet another computer that is optimized for processing data may be used to process information received from DMF instrument 105.

In DMF system 100, illumination source 154 and optical measurement device 156 may be arranged with respect to the portion of DMF cartridge 110 at which plasmonic particle assisted ELISA 112 occurs. The illumination source 154 may be, for example, a light source for the visible range (400-800 nm), such as, but not limited to, a white light-emitting diode (LED), a halogen bulb, an arc lamp, an incandescent lamp, lasers, and the like. Illumination source 154, in some embodiments, may be a “smart” bulb, capable of being operated and controlled via a mobile device. In addition to different wave lengths (i.e. different colors), the brightness may also be adjusted via the mobile device. Illumination source 154 is not limited to a white light source. Illumination source 154 may be any color light that is useful in DMF system 100. Optical measurement device 156 may be used to obtain light intensity readings. Optical measurement device 156 may be, for example, a charge coupled device, a photodetector, a spectrometer, a photodiode array, a smartphone camera, or any combinations thereof. Further, DMF system 100 is not limited to one illumination source 154 and one optical measurement device 156 only. DMF system 100 may include multiple illumination sources 154 and/or multiple optical measurement devices 156 to support multiple sensing elements. Thermal control mechanisms 158 may be any mechanisms for controlling the operating temperature of DMF cartridge 110. Examples of thermal control mechanisms 158 may include Peltier elements and resistive heaters.

Referring still to DMF system 100 of FIG. 1, by incorporating plasmonic particles into ELISA (i.e., plasmonic particle assisted ELISA 112), increased sensitivity and lower detection limits may be attained compared with conventional dyes (e.g., 3,3′,5,5′-tetramethylbenzidine (TMB)). Integrating the plasmonic particle-based ELISA into a digital microfluidic device (e.g., DMF cartridge 110) allows for DMF system 100 to be portable and be used for point-of-care (POC) applications. DMF system 100 and DMF cartridge 110 may also be used to selectively detect multiple proteins (e.g., antibodies IgG and IgM). Selective detection of multiple proteins may be achieved through using multi-modality plasmonic sensors.

DMF system 100 may be used for the detection and quantification of, for example, proteins (e.g., antibodies), viruses, bacteria, and/or any other pathogens proteins in DMF cartridge 110 using plasmonic particle assisted ELISA 112. Further, proteins may refer to both unbound proteins (e.g., after cell lysis) and proteins on the outer surface of cells and pathogens (e.g., viruses and bacteria). Using plasmonic particle assisted ELISA 112, plasmonic particles of various shapes, sizes, and compositions, which display tunable localized surface plasmon resonance peaks and/or fluorescence quenching through FRET may be processed. The plasmonic particles may be integrated into a digital microfluidic ELISA scenario in which the particles may undergo etching, growth, and/or aggregation in response to selective detection of proteins (e.g., antibodies). Etching, growth, and aggregation of the particles results in changes in the light absorbance, scattering, extinction and/or fluorescence quenching properties of the particles that can be detected with the naked eye and/or with instrumentation (e.g., a smartphone camera, which is one example of optical measurement device 156).

Generally, in DMF system 100 and/or DMF cartridge 110, plasmonic particle assisted ELISA 112 may be used to detect and quantify proteins (e.g., antibodies) in a sample. The plasmonic particles described herein may include nanoparticles of any type, shape, size, and material composition. In one example, the plasmonic particles are nanoparticles wherein one or more of the particle dimensions should be less than about 100 nm. In another example, for particles (e.g., nanoparticles) that include more than one material composition (e.g., core-shell particles) at least one of the particle layers must consist of a plasmonic layer. In yet another example, aside from the plasmonic layer, the particles (e.g., nanoparticles) may also include a dielectric, semiconductor, or polymeric material, and/or any other type of material. For example, in plasmonic particles having a semiconductor layer, the semiconductor layer may be magnetic, paramagnetic or superparamagnetic. In still another example, particle types (e.g., nanoparticles) that can be used with this disclosure may include, but are not limited to, core-shell particles, rattle-type particles, hollow particles, porous particles, bimetallic particles, and particles consisting of a single metal composition.

In DMF system 100 and/or DMF cartridge 110 and using plasmonic particle assisted ELISA 112, analyte concentration in samples may be quantified through colorimetric detection. Further, colorimetric change may be observed through the naked human eye and/or with instrumentation. For example, the colorimetric change or color change described herein, may include, but is not limited to, a change in the intensity of a color and/or perceivable color hues.

In DMF system 100 and/or DMF cartridge 110 and using plasmonic particle assisted ELISA 112, the selection of particles to be integrated in the device may be optimized for colorimetric detection using the naked human eye and/or using instrumentation (e.g., a smartphone camera, which is one example of optical measurement device 156). The initial and/or final readout color from the device should have a maximum fluorescence, absorbance, scattering, and or extinction peak of from about 380 nm to about 800 nm for colorimetric detection by the naked human eye. Further, the initial and/or final readout color from the device should have a maximum fluorescence, absorbance, scattering, and or extinction peak in the ultraviolet, visible, and/or infrared wavelengths for colorimetric detection with instrumentation.

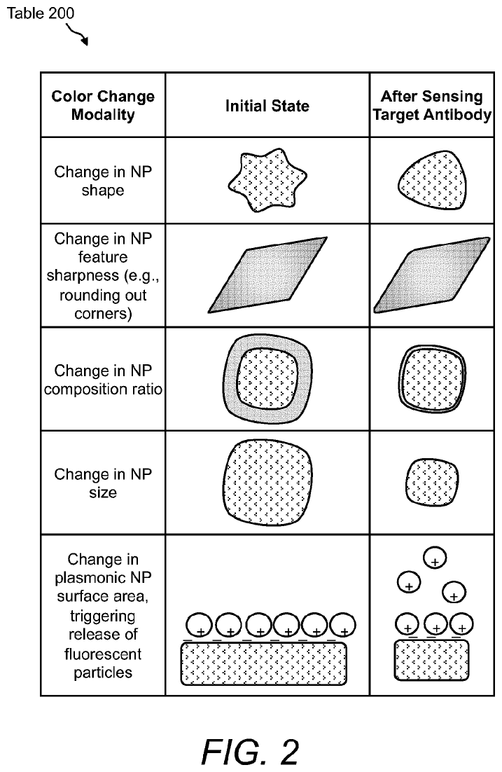

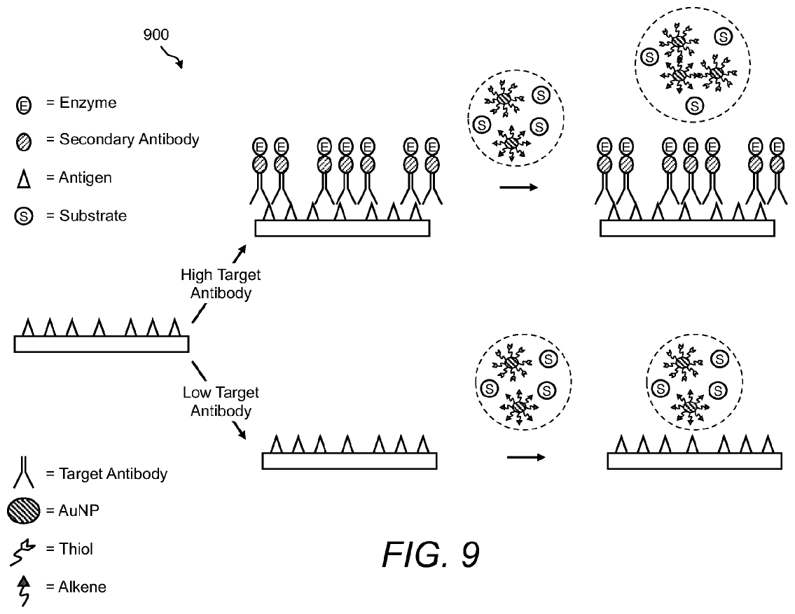

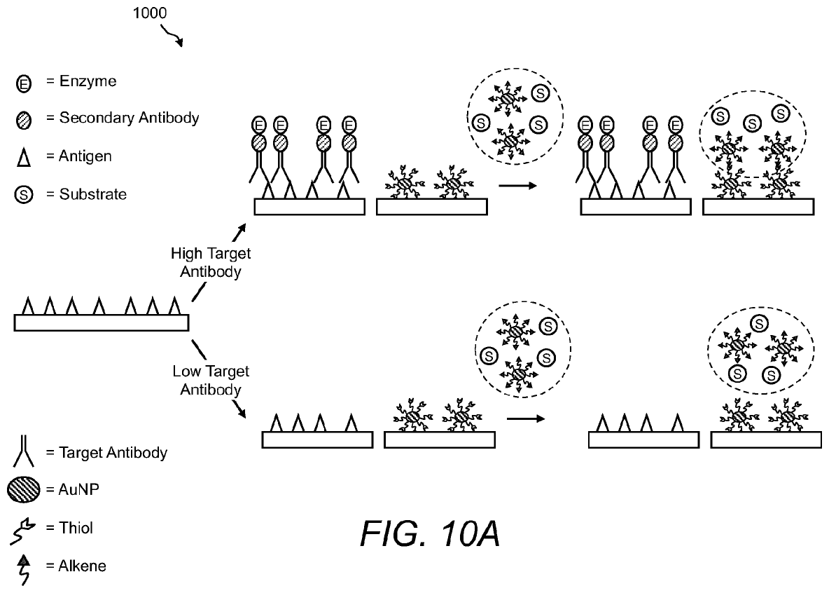

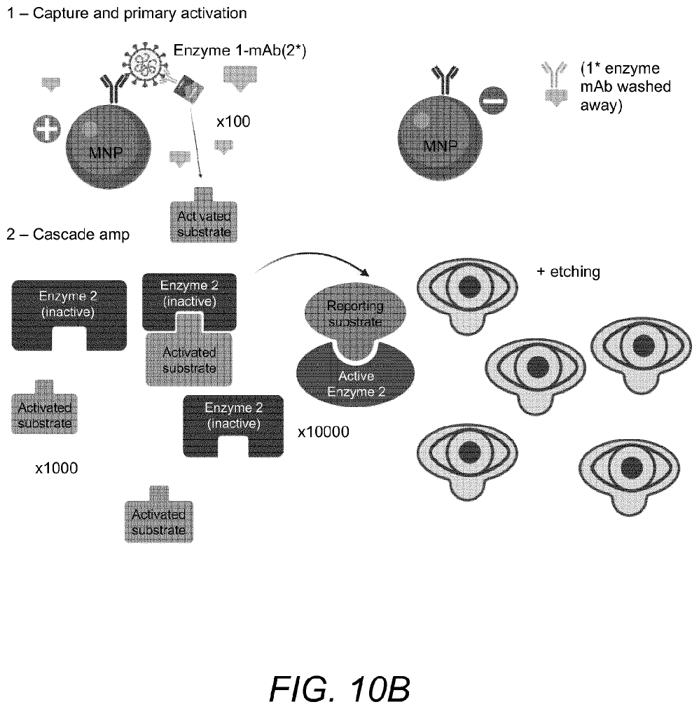

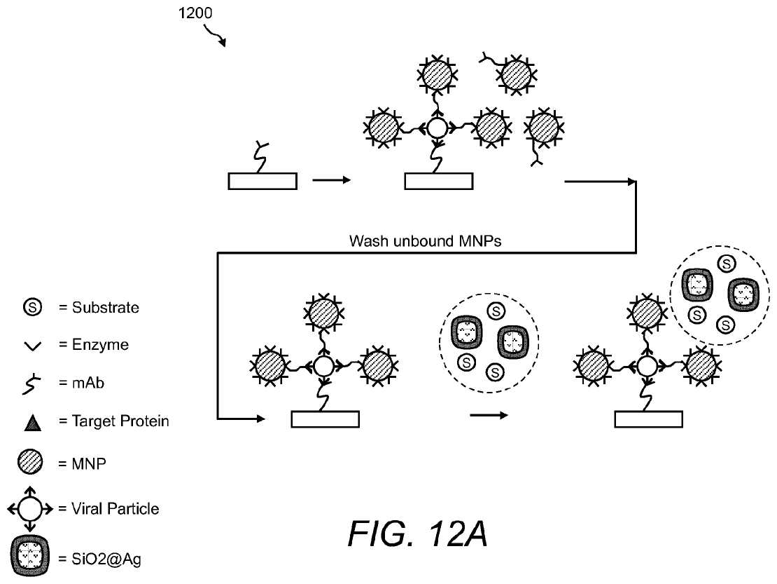

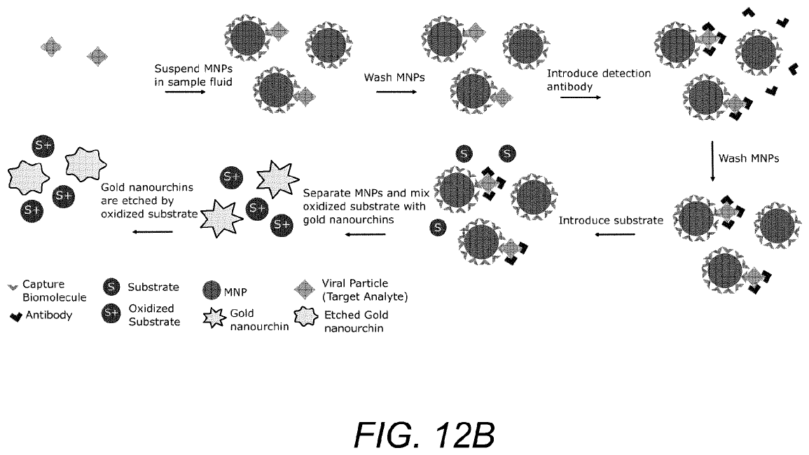

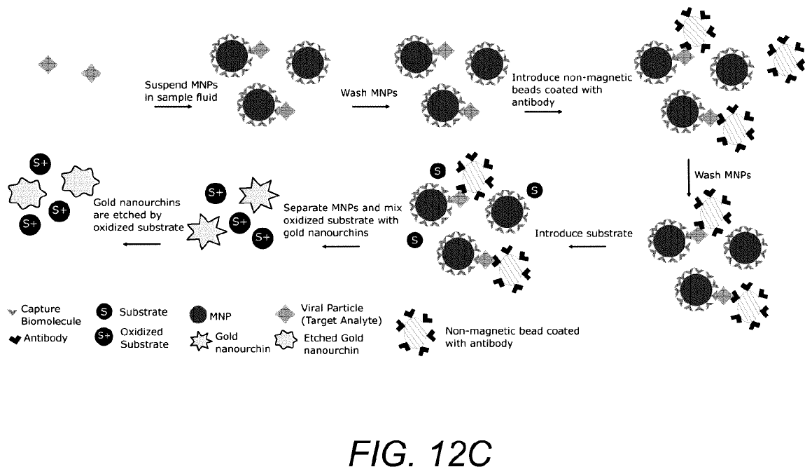

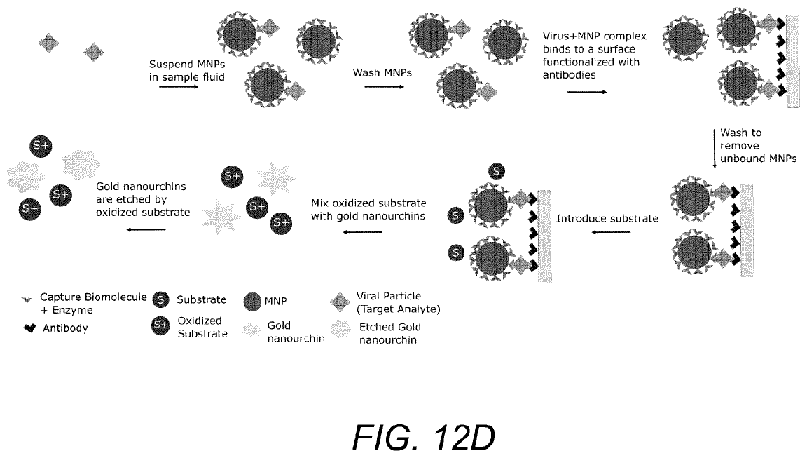

In DMF system 100 and/or DMF cartridge 110 and using plasmonic particle assisted ELISA 112, colorimetric change may be observed due to one of several scenarios.
(1) In one example, colorimetric change may occur due to etching of plasmonic particles (see FIG. 2 through FIG. 8D, FIG. 10B, and FIGS. 12B-12D).
(2) In another example, colorimetric change may occur due to the aggregation of plasmonic particles (see FIG. 9).
(3) In yet another example, colorimetric change may occur due to the growth of plasmonic particles.
(4) In yet another example, colorimetric change may occur due to the nucleation and growth of plasmonic particles by reducing metal ion precursors.
(5) In still another example, colorimetric change may occur due to quenching and/or unquenching the fluorescence of fluorescent probes (e.g., quantum dots) (see FIG. 10A).

In colorimetric change scenario #1 and using plasmonic particle assisted ELISA 112 of DMF system 100, colorimetric change may be observed due to etching of plasmonic particles by one or more of the modalities shown in Table 200 of FIG. 2. Table 200 of FIG. 2 indicates an example of color change modalities as plasmonic particles are etched in ELISA.

For example, Table 200 shows that etching of plasmonic particles changes the shape of the particles which shifts the LSPR peak of the particles. Table 200 also shows that etching of plasmonic particles changes the particle feature sharpness which shifts the LSPR peak of the particles. For example, rounding out sharp vertices is an example of changing particle feature sharpness. Table 200 also shows that etching of plasmonic particles changes the material composition ratio of a particle that includes more than one layer and/or material (e.g., silver coated gold nanospheres) which shifts the LSPR peak. Table 200 also shows that etching of plasmonic particles changes the size of the particles which shifts the LSPR peak. Table 200 also shows that etching of plasmonic particles reduces the surface area of the particles which allows fluorescent probes (e.g., quantum dots) that were adhered or chemically bound to the particles to be released or detached from the plasmonic particles. For example, the fluorescence of the fluorescent probes is quenched when they are bound to the plasmonic particles (or within a short distance from the surface of the plasmonic particles) through a mechanism known as FRET.

Continuing colorimetric change scenario #1 and using plasmonic particle assisted ELISA 112 of DMF system 100, the etching of plasmonic particles due to ELISA occurs as a result of oxidizing byproducts produced during enzyme-substrate reactions within the device. For example, if the enzyme is directly or indirectly linked to the target antibody and/or capture biomolecule, the amount of enzyme-substrate reactions is proportional to the number of target antibodies bound to capture biomolecules (e.g., antigens) in the device.

Examples 1-4 provided herein describe signal enhancement and limit of detection improvement in plasmonic nanoparticle assisted ELISA in a fluidics device. More specifically, Example 1 describes a high sensitivity sensor for H₂O₂ using TMB⁺-mediated etching of gold nanomaterials. Example 2 describes camera detection of gold nanoparticle etching by oxidized TMB on a DMF. Example 3 shows that biotin-HRP conjugated magnetic beads can oxidize TMB substrate on a DMF cartridge, and the oxidization of TMB can be detected by gold nanourchin etching, which results in a colorimetric change that can be measured by tracking the maximum LSPR peak position of the gold nanourchins. Example 4 describes a plasmonics assisted ELISA for detecting spike protein that includes capturing the spike protein (target analyte”) with anti-spike antibody conjugated magnetic beads (capture biomolecule), labelling the bead-spike protein complex with secondary anti-spike HRP (antibody having conjugated enzyme), incubating the beads with TMB (substrate), followed by etching of gold nanourchins by the oxidized TMB, which results in a colorimetric change that can be measured by tracking the maximum LSPR peak position of the gold nanourchins.

Continuing colorimetric change scenario #1 and using plasmonic particle assisted ELISA 112 of DMF system 100, the colorimetric change due to etching may be observed by one or more types of particles. An example of colorimetric detection due to etching multiple particles types is colorimetric plasmonic ELISA that is based on etching a dual particle system.

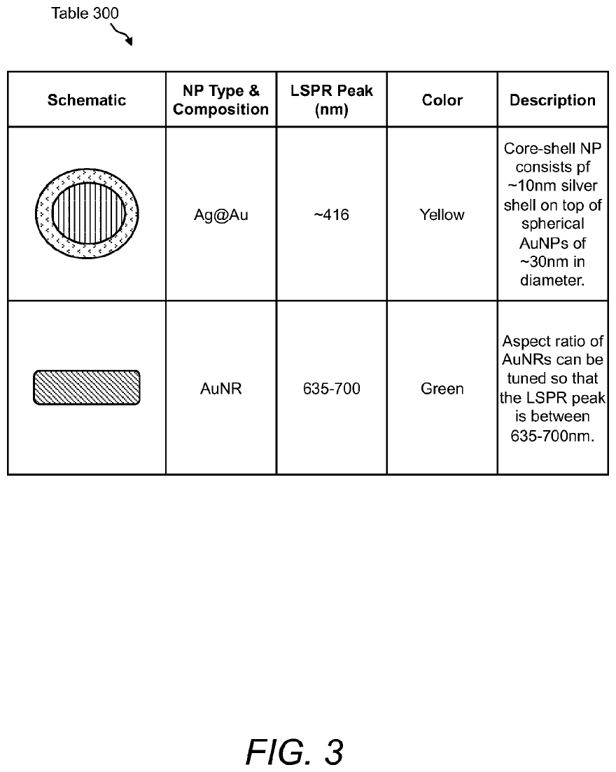

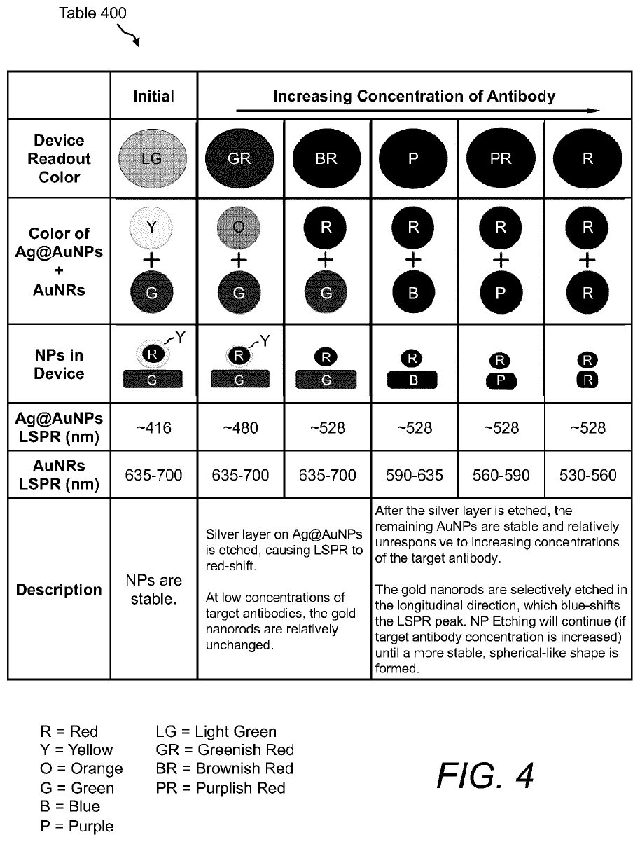

For example, a combination of two or more particle types may be used to increase the sensitivity and/or range of detection for a specific target antibody. Further, the number of perceivable color hues (by the naked human eye and/or with instrumentation) over the target antibody detection range using multiple particle types may be greater than the number of perceivable color hues using one of the particle types. Further to the example, a Table 300 shown in FIG. 3 indicates an example of properties of spherical Ag@Au core-shell particles and gold nanorods. Additionally, Table 400 shown in FIG. 4 indicates an example of colorimetric plasmonic ELISA based on etching a dual particle system.

Additionally, two different hues may be achieved by combining different aggregation states of the same particle. For example, combining an NP population as single particles and a second population of a controlled aggregation state (e.g., a grouping of 6× nanoparticles) may result in two distinct LSPR peaks, one being red and another being blue.

Continuing colorimetric change scenario #1 and using plasmonic particle assisted ELISA 112 of DMF system 100, the colorimetric change due to etching may be observed by one or more aggregation states of one or more types of particles. For example, a combination of aggregation states of one or more particle types may be used to increase the sensitivity and/or range of detection for a specific target antibody. Further, the number of perceivable color hues (by the naked human eye and/or with instrumentation) over the target antibody detection range using multiple particle aggregation states may be greater than the number of perceivable color hues using one particle aggregation state.

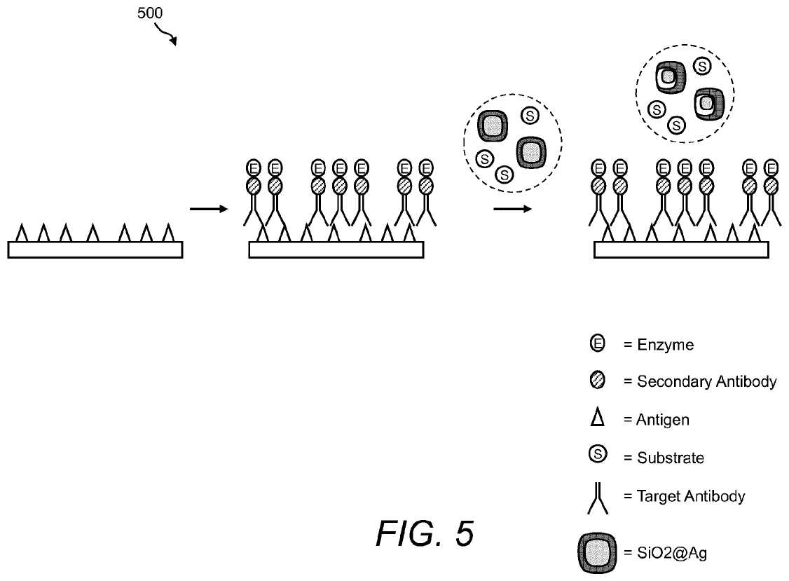

Continuing colorimetric change scenario #1 and using plasmonic particle assisted ELISA 112 of DMF system 100, the plasmonic particles may be suspended in solution during etching. Plasmonic particles may be transported in a fluidic suspension (or in a droplet) with the substrate to the site (in the device) where target antibodies are present. In one example, FIG. 5 shows a schematic diagram of an example of an etching-based plasmonic ELISA process 500 in a fluidics device in which colorimetric particles (e.g., NPs) are in suspension. In another example, the substrate and plasmonic particles may be transported as separate fluidic suspensions (or droplets) to the site where target antibodies are present.

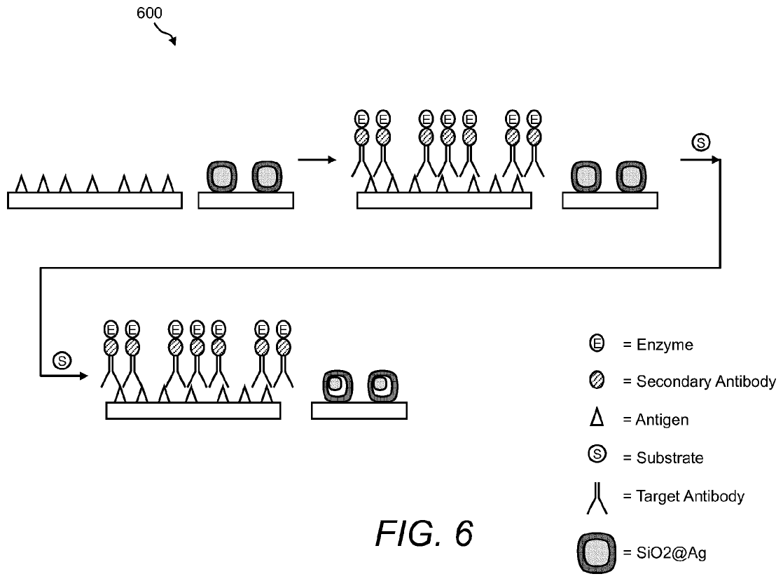

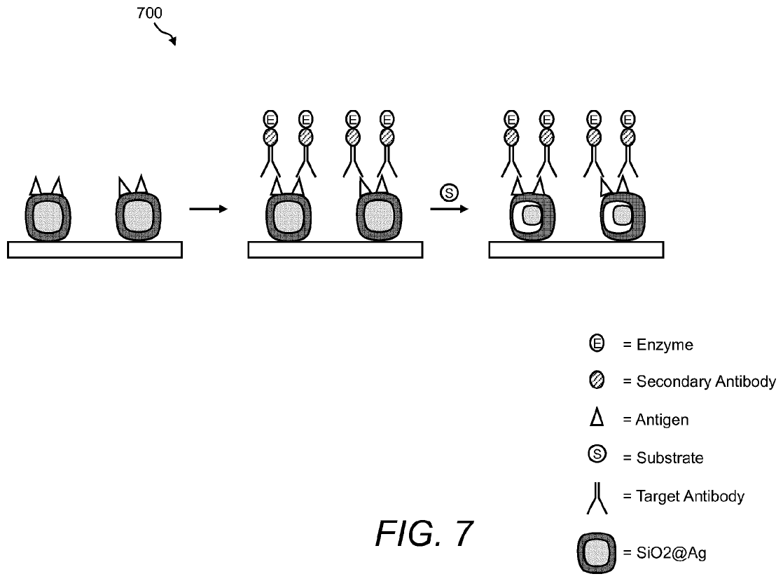

Continuing colorimetric change scenario #1 and using plasmonic particle assisted ELISA 112 of DMF system 100, the plasmonic particles may be immobilized on a surface during etching as a result of enzyme-substrate reactions during ELISA. Capture biomolecules that selectively bind the target antibodies may be immobilized on the same surface or on a separate surface as shown in FIG. 6. FIG. 6 shows a schematic diagram of an example of an etching-based plasmonic ELISA process 600 in a fluidics device in which colorimetric particles (e.g., NPs) are immobilized on a surface. Further, capture biomolecules that selectively bind the target antibodies may be chemically bound or adhered to the outer shell of a core-shell plasmonic particle as shown in FIG. 7. FIG. 7 shows a schematic diagram of an example of an etching-based plasmonic ELISA process 700 in a fluidics device in which colorimetric particles (e.g., NPs) are immobilized on a surface and antigens are bound to the plasmonic particles. The shell in the core-shell plasmonic particle will be porous (or mesoporous) to enable etching of the plasmonic core. Further, capture biomolecules that selectively bind the target antibodies may be suspended in solution.

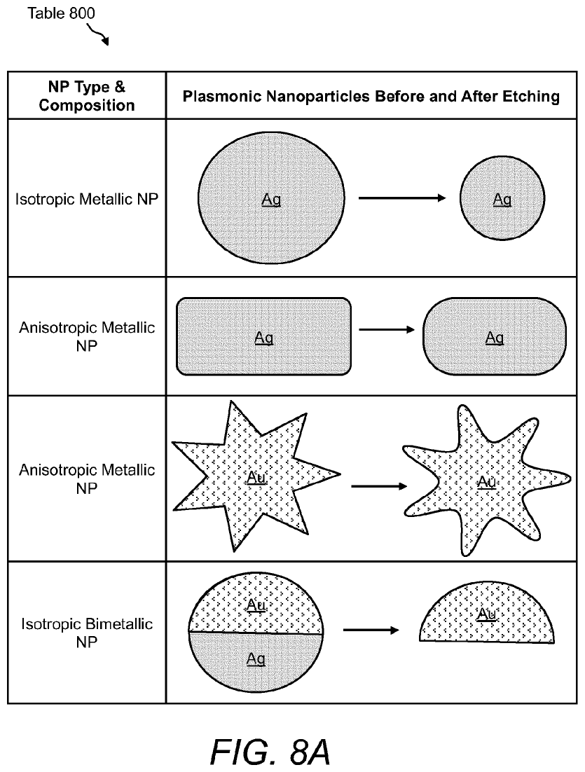

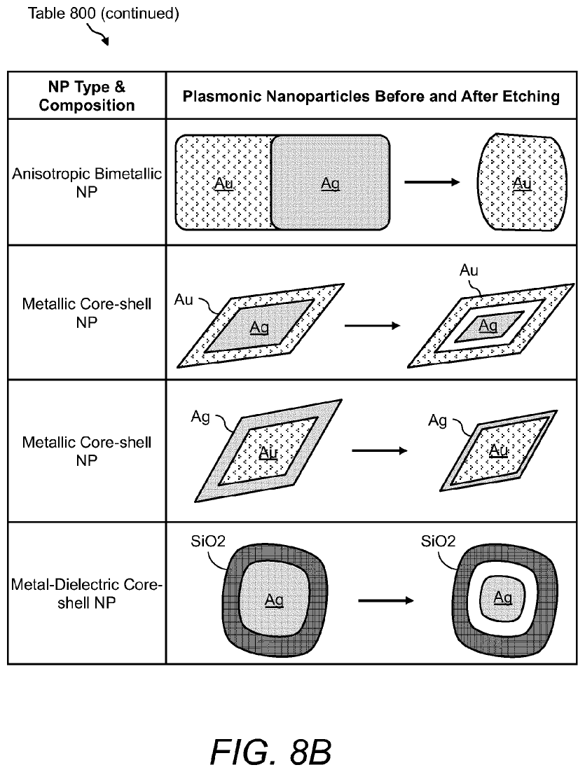

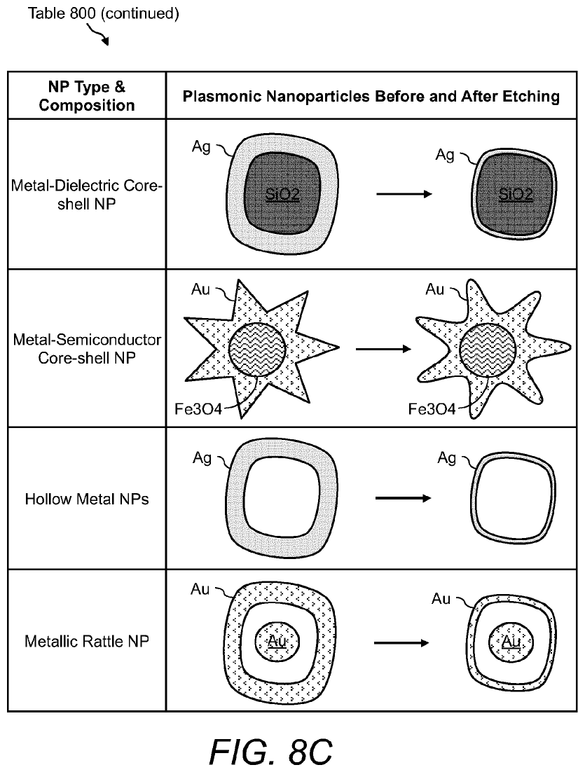

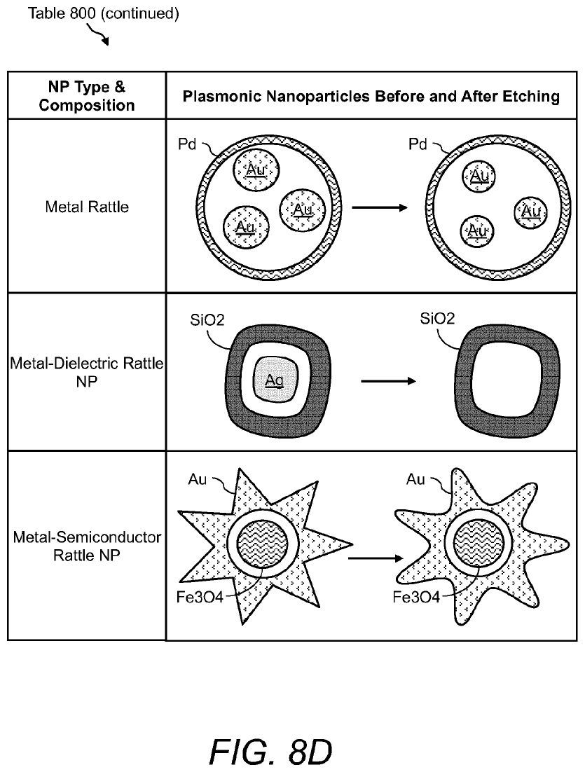

Continuing colorimetric change scenario #1 and using plasmonic particle assisted ELISA 112 of DMF system 100, one or more layers, features, and/or components of the particles may be etched as a result of enzyme-substrate reactions during ELISA. A Table 800 of FIG. 8A, FIG. 8B, FIG. 8C, and FIG. 8D indicates some examples of etching plasmonic particles (e.g., nanoparticles).

In colorimetric change scenario #2 and using plasmonic particle assisted ELISA 112 of DMF system 100, colorimetric change may be observed due to the aggregation of plasmonic particles or by reducing the interparticle distance. Particle aggregation and changes in the interparticle distance is due to modulating the attractive or repulsive forces among particles which is catalyzed by enzyme-substrate reactions during ELISA in the device. Further, if the enzyme is directly or indirectly linked to the target antibody and/or capture biomolecule, the amount of enzyme-substrate reactions is proportional to the number of target antibodies bound to capture biomolecules (e.g., antigens) in the device.

Additionally, particle aggregation (and also etching) may be provided in the presence of another background color, created by a dye or another population of particles which does not aggregate or etch (etching or lack of aggregation can perhaps be prevented by a protective surface modification). Oftentimes, mass aggregation results in more of a loss of color compared with a change in color. So here, as aggregation/etching of the particle increases, it's particular color absorbance peak may disappear, changing the hue to the constant color present (that which doesn't change).

Continuing colorimetric change scenario #2 and using plasmonic particle assisted ELISA 112 of DMF system 100, changes in the interparticle distance may be stimulated by one or more mechanisms. These mechanisms may include, but are not limited to, hydrogen bonding between particles, decreasing the surface charge of particles, conjugating particles together through chemical reactions (e.g., cycloaddition), and by bridging particles together through bioconjugation.

Continuing colorimetric change scenario #2 and using plasmonic particle assisted ELISA 112 of DMF system 100, the surface charge associated with plasmonic particles may be modified by ionic molecules containing one or more functional groups that can covalently bind to plasmonic particles (e.g., carboxyl, thiol, and/or amine groups). The addition or presence of these functional groups is catalyzed by enzyme-substrate reactions during ELISA.

Continuing colorimetric change scenario #2 and using plasmonic particle assisted ELISA 112 of DMF system 100, the interparticle distance between plasmonic particles may be tuned by enzyme-catalyzed chemical bonding between plasmonic particles with different functional groups. This may include, but is not limited to, click reactions, such as thiol-ene and copper(I)-catalyzed azide-alkyne cycloaddition. In one example, FIG. 9 is a schematic diagram of an example of an aggregation-based plasmonic ELISA process 900 in a fluidics device.

In colorimetric change scenario #3 and using plasmonic particle assisted ELISA 112 of DMF system 100, colorimetric change may be observed due to the growth of plasmonic particles catalyzed by the detection of target antibodies using ELISA in the device. Growth of plasmonic particles due to ELISA occurs as a result of reducing metal ion precursors catalyzed by enzyme-substrate reactions within the device. The amount of enzyme-substrate reactions is proportional to the number of target antibodies bound to capture biomolecules (e.g., antigens) in the device.

Continuing colorimetric change scenario #3 and using plasmonic particle assisted ELISA 112 of DMF system 100, colorimetric change due to growth of plasmonic particles may be observed by one or more types of particles.

Continuing colorimetric change scenario #3 and using plasmonic particle assisted ELISA 112 of DMF system 100, core-shell particles may be formed due to reduction of metal ion precursors, followed by growth of a thin shell on top of a plasmonic (or non-plasmonic) core.

Continuing colorimetric change scenario #3 and using plasmonic particle assisted ELISA 112 of DMF system 100, metal ion precursors may be reduced and deposited on plasmonic cores or shells containing the same metallic material.

Continuing colorimetric change scenario #3 and using plasmonic particle assisted ELISA 112 of DMF system 100, particle growth may result in uniform growth of all crystal facets, directional growth, and/or anisotropic growth.

Continuing colorimetric change scenario #3 and using plasmonic particle assisted ELISA 112 of DMF system 100, particles may be suspended in solution during enzyme-catalyzed growth. Particles may be transported in a fluidic suspension (or in a droplet) with the substrate, metal ion precursors, and other reagents to the site (in the device) where target antibodies are bound. In another example, the substrate, particles, metal ion precursors, and other reagents may be transported as separate fluidic suspensions (or droplets) to the site where target antibodies are bound.

Continuing colorimetric change scenario #3 and using plasmonic particle assisted ELISA 112 of DMF system 100, enzyme-catalyzed growth can occur on particles that are immobilized on a surface. For example, particles and capture biomolecules that selectively bind the target antibodies may be immobilized on the same surface or on a separate surface.

In colorimetric change scenario #4 and using plasmonic particle assisted ELISA 112 of DMF system 100, colorimetric change may occur due to the nucleation and growth of plasmonic particles catalyzed by the detection of target antibodies using ELISA in the device. Nucleation and growth of plasmonic particles due to ELISA occurs as a result of reducing metal ion precursors catalyzed by enzyme-substrate reactions within the device. The amount of enzyme-substrate reactions is proportional to the number of target antibodies bound to capture biomolecules (e.g., antigens) in the device. This differs from colorimetric change scenario #3 that also describes colorimetric change due to the growth of plasmonic particles catalyzed by the detection of target antibodies using ELISA in that here the nucleation of new particles occurs within the device after enzyme-substrate reactions, rather than growth on pre-formed particles.

Continuing colorimetric change scenario #4 and using plasmonic particle assisted ELISA 112 of DMF system 100, nucleation and growth of plasmonic particles may also take place within the device at zero or low concentrations of bound target antibodies. However, the colorimetric response at zero or low concentrations of bound target antibodies may be clearly distinguished from the colorimetric response at higher concentrations of bound target antibodies by the naked eye and/or with instrumentation.

In colorimetric change scenario #5 and using plasmonic particle assisted ELISA 112 of DMF system 100, colorimetric change may occur due to a change in the fluorescence from fluorescent probes (e.g., quantum dots) catalyzed by the detection of target antibodies using ELISA in the device. Fluorescence modulation by ELISA may be achieved by tuning the distance between fluorescent probes and plasmonic particles, which is mediated by enzyme-substrate reactions within the device. The amount of enzyme-substrate reactions is proportional to the number of target antibodies bound to capture biomolecules (e.g., antigens) in the device. Further, the fluorescence from fluorescent probes is quenched when they are within a short distance from the surface of plasmonic particles through a mechanism known as FRET. Further, the fluorescence intensity from fluorescent probes is higher when the fluorescent probes are not within a short distance from the surface of plasmonic particles or from the surface of another fluorescence quencher/acceptor moiety (e.g., a “black hole quencher”). An example of this type of actuation may be short oligomer (e.g., peptide or aptamer or carbohydrate-based molecules) that are dual-labeled by a FRET pair for enzymatically amplified cascade assay.

Continuing colorimetric change scenario #5 and using plasmonic particle assisted ELISA 112 of DMF system 100, changes in the distance between plasmonic particles and fluorescent probes may be stimulated by one or more mechanisms. These mechanisms may include, but are not limited to, hydrogen bonding between particles and/or fluorescent probes, decreasing the surface charge of particles and/or fluorescent probes, conjugating particles and/or fluorescent probes together through chemical reactions (e.g., cycloaddition), and by bridging particles and/or fluorescent probes together through bioconjugation.

Continuing colorimetric change scenario #5 and using plasmonic particle assisted ELISA 112 of DMF system 100, the surface charge associated with plasmonic particles and/or fluorescent probes may be modified by ionic molecules containing one or more functional groups that can covalently bind to plasmonic particles and/or fluorescent probes (e.g., carboxyl, thiol, and/or amine groups). The addition or presence of these functional groups is catalyzed by enzyme-substrate reactions during ELISA.

Continuing colorimetric change scenario #5 and using plasmonic particle assisted ELISA 112 of DMF system 100, the distance between plasmonic particles and fluorescent probes may be tuned by enzyme-catalyzed chemical bonding. This may include, but is not limited to, click reactions such as thiol-ene and copper(I)-catalyzed azide-alkyne cycloaddition. For example, FIG. 10A is a schematic diagram of an example of a FRET-based plasmonic ELISA process 1000 in a fluidics device. In another example, mAb-conjugated (specific against target) enzyme 1 directly activates an enzyme 2 in a dose dependent manner, which then converts the reporter molecule (or produces the recorded output) in a greatly magnified quantity. Enzyme cascades can be either “traditional” substrates (i.e., ALP with phosphates, HRP with TMB, or GOx w/Glu) or they can be proteolytic cascades (e.g., the thrombin pathway or glycolytic (e.g., glycogen phosphorylase, amylase, etc.)).

FIG. 10B is a schematic diagram showing an example of a process of colorimetric change by enzyme catalyzed cascade that can be performed as a plasmonic nanoparticle assisted ELISA process in a fluidic device in which the activity of a first enzyme in the presence of the target analyte activates a second “reporting” enzyme that produces a product that etches a plasmonic nanoparticle. In FIG. 10B, Step 1 illustrates capture of the target virus (analyte), which in this case is a COVID virus, onto monoclonal antibody (mAb) (the mAb is the “capture biomolecule”) coated magnetic nanoparticles (MNPs) otherwise referred to as “beads”. The beads are washed and a second anti-COVID mAb (“antibody”) having a distinct epitope is introduced and binds to the immobilized virus. In the next step, an anti-2^{nd} mAb is introduced which is specific against the second mAb and is conjugated with Enzyme 1. Next, Step 2—Cascade amp illustrates that an Enzyme 2 is introduced which is activated by Enzyme 1 (amplification value has to be at least 10:1). At this point the droplet containing Enzyme 2 (which is now partially active proportionally to the quantity of virus via the preceding steps) is combined with a substrate droplet for this Enzyme-2. The results of this process can have a wide dynamic range (orders of magnitude) due to the nature of enzymatic reactions.

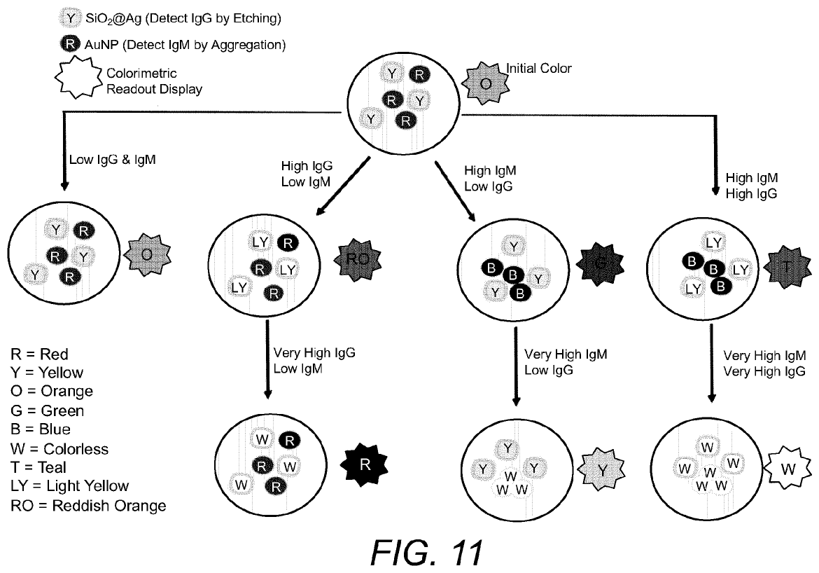

Referring now again to FIG. 1, DMF system 100 and/or DMF cartridge 110 that includes plasmonic particle assisted ELISA 112 may be used to selectively detect and quantify the concentration of more than one type of protein with high specificity, using the processes described hereinabove. Further, one or more types of particles may be used to detect multiple target proteins. Colorimetric detection and quantification of multiple target proteins may be monitored using one or more readout displays. More than one of the sensing modalities may be used to detect multiple target proteins with high specificity. For example, FIG. 11 is a schematic diagram of an example of a process 1100 of colorimetric detection of multiple proteins in a fluidics device using multimodality plasmonic particle sensors (i.e., particles with different sensing modalities). In process 1100, the detection and quantification of multiple target proteins is observed in one colorimetric readout display.

In DMF system 100 and/or DMF cartridge 110 and using plasmonic particle assisted ELISA 112, the assay detection limit may be decreased through the use of multi-functionalized particles (e.g., nanoparticles or microparticles) containing capture biomolecules to selectively detect target proteins and multiple reporter enzymes to amplify the colorimetric response. The multi-functionalized particles may contain a magnetic core (or layer), thereby allowing their movement within the fluidic device to be controlled by an external magnetic field. The multi-functionalized particles may contain a plasmonic layer. The ratio of reporter enzymes to capture biomolecules on the multi-functionalized particles may be greater than 1. The multi-functionalized particles may contain functional groups other than reporter enzymes and capture biomolecules.

In DMF system 100 and/or DMF cartridge 110 and using plasmonic particle assisted ELISA 112, target proteins on the outer surface of pathogenic particles (e.g., virus particles) can bind to multiple multi-functionalized particles (described above) and in some examples to another surface functionalized with second capture biomolecules (e.g., monoclonal antibodies), wherein the second capture biomolecule is also referred to herein for the purposes of the specification and claims as an antibody that binds to a site on the target analyte that is different than the capture biomolecule. The second capture biomolecule or antibody may be immobilized on another surface referred to herein for the purposes of the specification and claims as a second surface such as, for example, a non-magnetic bead or another non-magnetic surface. The ratio of multi-functionalized particles bound per pathogenic particle may be equal to or greater than 1. Colorimetric detection and quantification of pathogenic particles (and/or target proteins) may be achieved using the processes described hereinabove. Multi-functionalized particles that are not bound to pathogenic particles may be removed prior to introducing specific substrates that bind with the reporter enzymes on the multi-functionalized particles. For example, FIG. 12A is a schematic diagram of an example of a process 1200 of signal amplification in a fluidics device by binding one or many multi-functionalized magnetic particles to target viral particles.

In FIG. 12B, one example of an etching-based plasmonic ELISA process in a fluidics device is illustrated where the target is a viral particle, the capture biomolecule is attached to a magnetic nanoparticle (MNP) and the enzyme is conjugated to the antibody. In this example, the capture biomolecule is immobilized on a magnetic nanoparticle (MNP) and introduced to the target viral particle to form a target-capture biomolecule complex. After washing, an antibody that binds to the target at a different site than the capture biomolecule and that has a conjugated enzyme is introduced. After another washing, a substrate for the enzyme is introduced and the reaction in which the enzyme oxidizes the substrate is allowed to proceed. After the enzyme-substrate reactions, the magnetic beads are separated from the oxidized substrate and gold nanourchins in suspension are mixed with the oxidized substrate. The etching of the gold nanourchins in the presence of the oxidized substrate results in an optically detectable change that is measured, where the amount of enzyme-substrate reactions is proportional to the number of target analytes bound to the capture biomolecules.

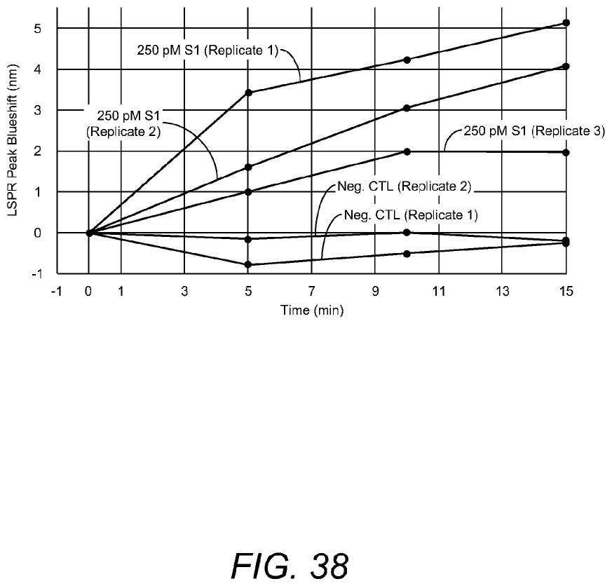

FIG. 38 is a graph showing the results of experimental implementation on a DMF device of the scheme shown in FIG. 12B where spike protein is the target analyte (see Example 4). FIG. 38 shows the maximum LSPR peak position of gold nanourchins blue-shifted significantly more for gold nanourchins mixed with TMB that had been incubated with beads mixed with 250 pM spike protein than for the negative control. This result shows that spike protein in solution can be detected by performing plasmonic ELISA on DMF. In this example, the spike protein plasmonic ELISA consists of capturing the spike protein (target analyte”) with anti-spike antibody conjugated magnetic beads (capture biomolecule), labelling the bead-spike protein complex with secondary anti-spike HRP (antibody having conjugated enzyme), incubating the beads with TMB (substrate), followed by etching of gold nanourchins by the oxidized TMB, which results in a colorimetric change that can be measured by tracking the maximum LSPR peak position of the gold nanourchins.

In FIG. 12C, one example of an etching-based plasmonic ELISA process in a fluidics device is illustrated where the target is a viral particle, the capture biomolecule is attached to a magnetic nanoparticle (MNP) and the antibody and the enzyme are attached to a non-magnetic bead. In this example, the capture biomolecule is immobilized on a magnetic nanoparticle (MNP) and introduced to the target viral particle to form a target-capture biomolecule complex. After washing, non-magnetic beads are introduced that have an attached antibody that binds to the target at a different site than the capture biomolecule and also contain an attached enzyme. After another washing, a substrate for the enzyme is introduced and the reaction in which the enzyme oxidizes the substrate is allowed to proceed. After the enzyme-substrate reactions, the magnetic beads are separated from the oxidized substrate and gold nanourchins in suspension are mixed with the oxidized substrate. The etching of the gold nanourchins in the presence of the oxidized substrate results in an optically detectable change that is measured, where the amount of enzyme-substrate reactions is proportional to the number of target analytes bound to the capture biomolecules.

In FIG. 12D, one example of an etching-based plasmonic ELISA process in a fluidics device is illustrated where the target is a viral particle, the capture biomolecule and enzyme are attached to a magnetic nanoparticle (MNP). In this example, the capture biomolecule is immobilized on a magnetic nanoparticle (MNP) and introduced to the target viral particle to form a target-capture biomolecule complex. After washing, the complex is introduced to a surface functionalized with an antibody that binds to the target at a different site than the capture biomolecule. After another washing, a substrate for the enzyme is introduced and the reaction in which the enzyme oxidized the substrate is allowed to proceed. After the enzyme-substrate reactions, the oxidized substrate is mixed with gold nanourchins. The etching of the gold nanourchins in the presence of the oxidized substrate results in an optically detectable change that is measured, where the amount of enzyme-substrate reactions is proportional to the number of target analytes bound to the capture biomolecules

In DMF system 100 and/or DMF cartridge 110 and using plasmonic particle assisted ELISA 112, target proteins can bind to capture biomolecules (e.g., monoclonal antibodies) on multi-functionalized particles (described above) and on an additional surface to form a structure similar to conventional sandwich ELISA. The capture biomolecules on the multi-functionalized particles and additional surface may bind different epitopes on the target protein or different target proteins on the viral particle. Further, pathogenic particles may be lysed so that sandwich structures consisting of capture biomolecules and the target protein contain a singular target protein per multifunctional particle. Lysing pathogenic particles may allow for detection of multiple target proteins per pathogenic particle, thereby amplifying the colorimetric response. For example, FIG. 13 is a schematic diagram of an example of a process 1300 of colorimetric signal amplification in a fluidics device by binding a single target protein to multi-functionalized magnetic nanoparticles containing multiple reporter enzymes.

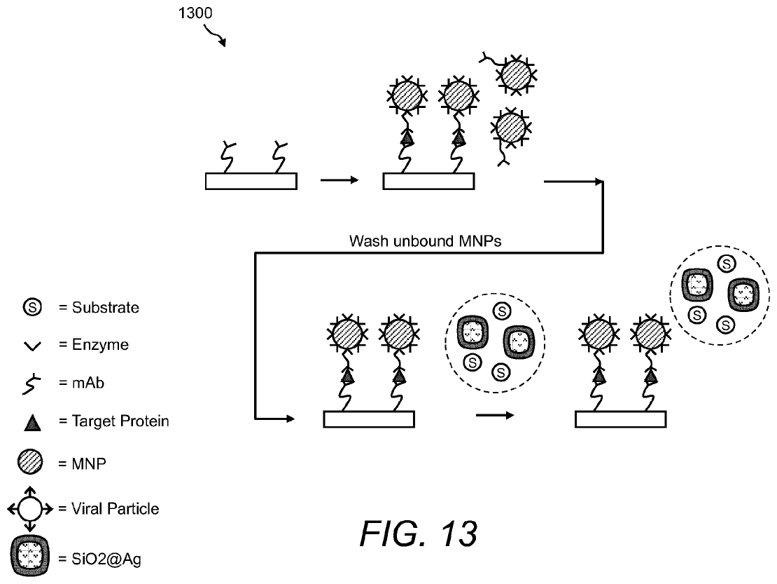

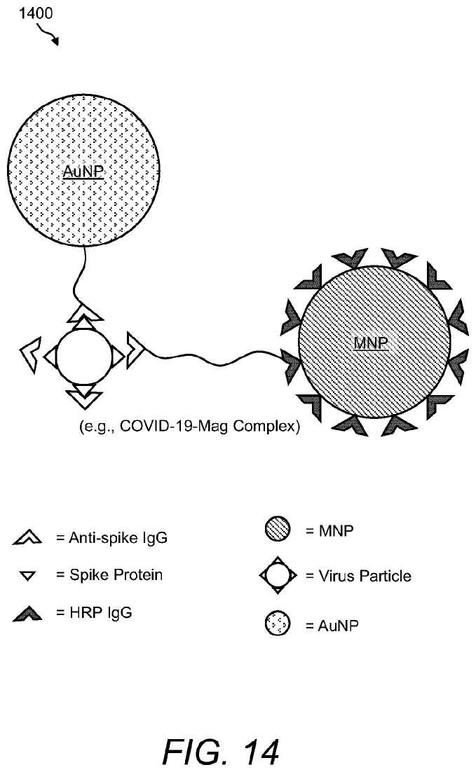

Referring now to FIG. 14 through FIG. 21 is schematic diagrams of examples of yet other processes of colorimetric change.

FIG. 14 is a schematic diagram of an example of a process 1400 of colorimetric change by etching gold nanoparticles. In process 1400, the AuNP may be coated in anti-spike antibody and binds to the MNP only when there is virus present. Then, when the TMB is introduced, the HRP present will convert it to TMB2+ (or TMB+) TMB2+, which then etches the AuNP and changes the color of the drop. However, when there is no virus present, the color of the drop will just be the color of TMB2+, i.e. yellow (or TMB+, i.e., blue). In summary, the virus is captured with the MNP (the capture biomolecule is conjugated to the MNP), then the HRP on the MNP produces oxidized TMB, and then oxidized TMB etches the AuNP. In this process, the MNP may be, for example, a 1.5-2 μg avidin/mg particle (50-60 molecules avidin/particle).

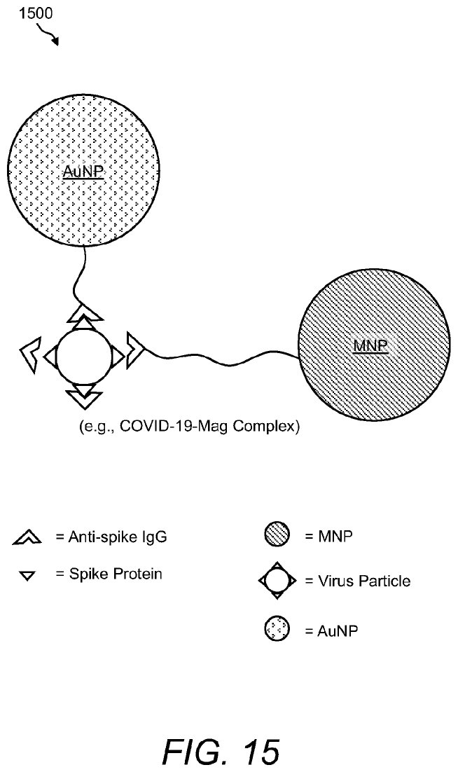

FIG. 15 is a schematic diagram of an example of a process 1500 of colorimetric change by color accumulation or depletion. Process 1500 may be like a magnetically assisted lateral flow assay in which the accumulation of color due to AuNPs binding to the virus-magnetic bead complex is being detected. When there is no virus present, the wash operation will yield magnetic beads only. However, when there is virus present, then the AuNP-magnetic bead complex is present, which will be a different color. This process may also be used in a depletion format wherein the AuNPs are captured from solution with the magnets. Then, the decrease in color due to loss of AuNPs is monitored. Again, in this process, the MNP may be, for example, a 1.5-2 μg avidin/mg particle (50-60 molecules avidin/particle).

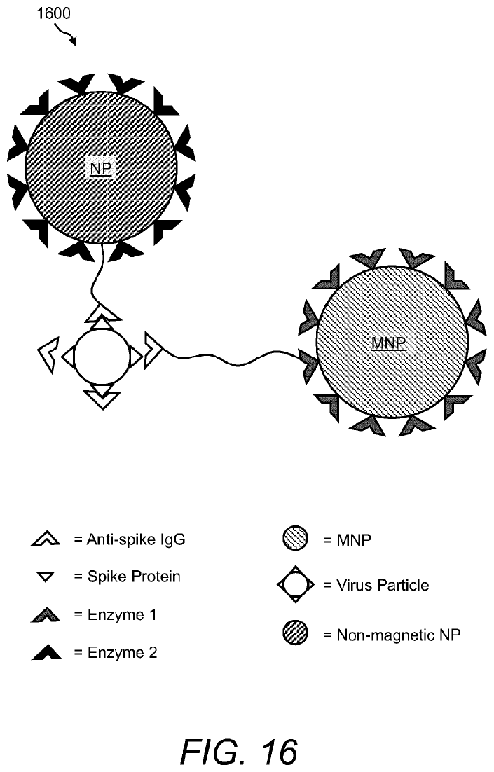

FIG. 16 is a schematic diagram of an example of a process 1600 of colorimetric change by enzyme catalyzed cascade. In process 1600, a MNP has a first enzyme (e.g., enzyme 1) on it and a non-magnetic nanoparticle (NP) (e.g., any non-magnetic bead) has a second enzyme (e.g., enzyme 2) on it. The product of enzyme 1 becomes the substrate for enzyme 2. Accordingly, the detection is done based on the presence of the product of enzyme 2. When there is virus present, the result is the product of enzyme 2. However, when there is no virus present, the result is the product of enzyme 1 only. A benefit of process 1600 is an enzyme cascade with a very high enzyme concentration. Again, in this process, the MNP may be, for example, a 1.5-2 μg avidin/mg particle (50-60 molecules avidin/particle).

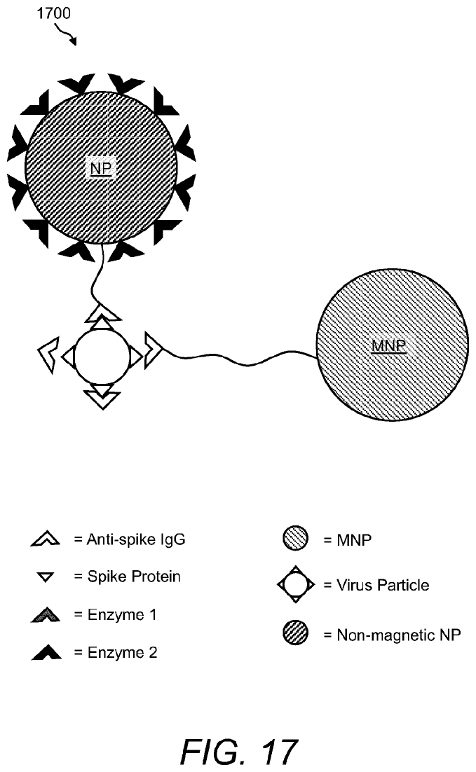

FIG. 17 is a schematic diagram of an example of a process 1700 of colorimetric change by HRP on a non-magnetic nanoparticle. In process 1700, the non-magnetic nanoparticle (NP) may be a bifunctional non-magnetic bead, such as a latex bead functionalized with HRP and anti-spike antibody. The non-magnetic NP may alternatively be a mono-functional bead with an HRP labeled anti-spike antibody. The other particle is a MNP with anti-spike antibody thereon. In the presence of virus, the non-magnetic NP is anchored to the MNP through binding of the viral spike protein. A washing operation is then performed and if virus is present, the non-magnetic NP remains anchored to the MNP and can be detected in a colorimetric reaction using HRP and TMB. If no virus is present, the non-magnetic NP bead is washed away and no color can be detected. Again, in this process, the MNP may be, for example, a 1.5-2 μg avidin/mg particle (50-60 molecules avidin/particle).

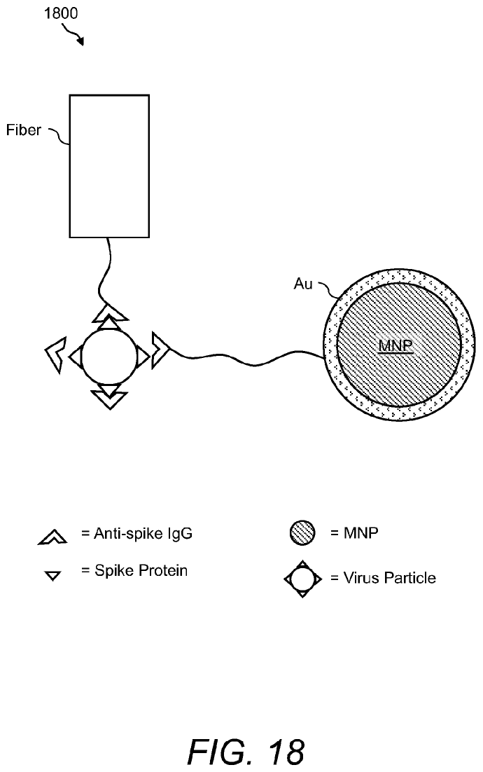

FIG. 18 is a schematic diagram of an example of a process 1800 of colorimetric change using a particle or bead that has a magnetic core and gold coating or shell. Accordingly, this particle or bead has both optical and magnetic properties. In process 1800, a fiber may be used to immobilize the magnetic core-gold shell bead. The accumulation of the bead or the depletion of the bead from a drop is monitored. Again, in this process, the MNP (i.e., the core) may be, for example, a 1.5-2 μg avidin/mg particle (50-60 molecules avidin/particle).

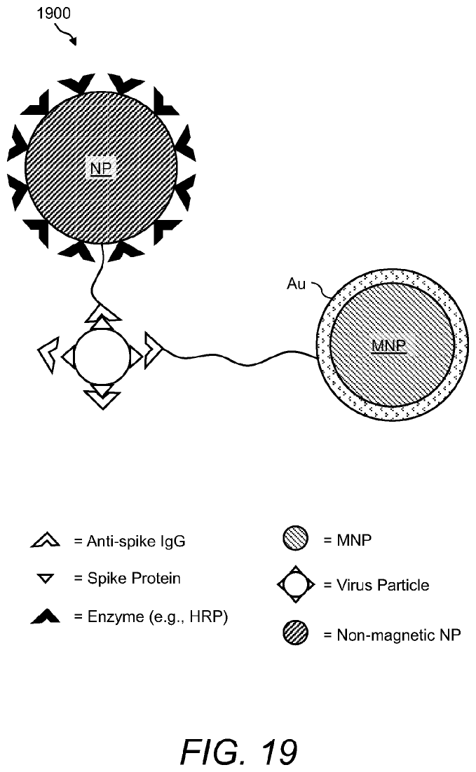

FIG. 19 is a schematic diagram of another example of a process 1900 of colorimetric change using a particle or bead that has a magnetic core and gold coating or shell. Accordingly, this particle or bead has both optical and magnetic properties. In process 1900, a non-magnetic NP is used that is bifunctional with HRP (or other enzyme that can generate a colorimetric response) and anti-spike antibody.

When there is virus present, the non-magnetic NP is anchored via the MNP and remains during a wash operation. When TMB is introduced, the HRP present on the non-magnetic NP will convert it to oxidized TMB (e.g., TMB2+) which then etches the gold shell on the MNP, changing the color. However, when virus is not present, no oxidized TMB is produced and therefore no etching will occur. Again, in this process, the MNP (i.e., the core) may be, for example, a 1.5-2 μg avidin/mg particle (50-60 molecules avidin/particle).

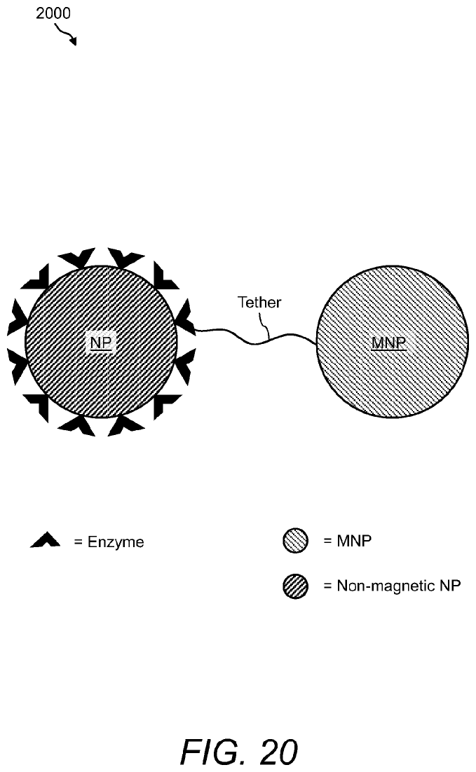

FIG. 20 is a schematic diagram of an example of a process 2000 of using plasmonic nanoparticle assisted ELISA 112 of DMF system 100 with respect to a CRISPR assay. FIG. 20 shows, for example, a tether linking an enzyme (e.g., HRP) labeled non-magnetic nanoparticle (NP) to a MNP. The MNP may be functionalized with a capture biomolecule (e.g., anti-spike antibody (IgG)) for binding and concentrating viral particles. The tether may be, for example, a nucleic acid sequence (e.g., a DNA sequence) that is targeted by a CRISPR enzyme for cleavage. Again, in this process, the MNP may be, for example, a 1.5-2 μg avidin/mg particle (50-60 molecules avidin/particle). Process 2000 may include, but is not limited to, the following steps.
(1) Magnetic bead preconcentration of the virus, wherein a MNP with anti-viral antibody (“capture biomolecule”) thereon is used in a preconcentration process to capture and concentrate viral particles present in a sample;
(2) Viral lysis, wherein viral particles are lysed thereby allowing for detection of multiple viral targets per pathogenic particle;
(3) Isothermal amplification of viral nucleic acid sequences (e.g., using a loop-mediated-isothermal amplification (LAMP) or a reverse transcriptase-LAMP (RT-LAMP) protocol);
(4) Detection of virus where in the presence of viral nucleic acid sequences, the CRISPR enzyme will cleave the tether thereby separating the enzyme labeled non-magnetic NP and the MNP. The enzyme labeled non-magnetic NP and the MNP particles can then be separated into two different droplets (e.g., during a wash operation). In one example, the presence of virus can then be detected by a colorimetric change in the enzyme labeled non-magnetic NP droplet. In another example, the presence of virus can be detected by a lack (depletion) of color in the MNP droplet. In yet another example, a colorimetric change in the enzyme labeled non-magnetic NP droplet and a lack (depletion) of color in the MNP droplet can be used to detect the presence of virus.

In another example of process 2000, the non-magnetic NP may be replaced with an AuNP and the presence of virus can be detected using the accumulation/depletion of color in a process similar to a lateral flow assay (e.g., as described above with reference to FIG. 15).

In yet another example of process 2000, any of the aforementioned processes 1400, 1500, 1600, 1700, 1800, 1900 may be applied to process 2000 as the read out for detecting the presence (or absence) of virus. In this example, instead of two particles (e.g., NPs) becoming tethered together in the presence of virus, the two particles are separated in the presence of viral nucleic acid sequences by CRISPR enzyme mediated cleavage of a tether.

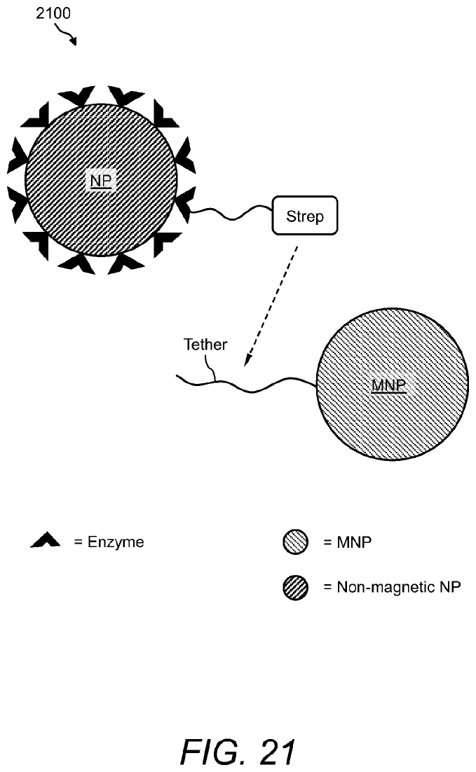

FIG. 21 is a schematic diagram of an example of a process 2100 of using plasmonic nanoparticle assisted ELISA 112 of DMF system 100 with respect to another CRISPR assay. FIG. 21 shows, for example, an enzyme (e.g., HRP) labeled non-magnetic nanoparticle (NP) that is functionalized with streptavidin. FIG. 21 also shows a MNP with a tether that includes a biotin tag. In this example, a CRISPR enzyme is used to cleave the end of the tether on the MNP and expose a biotin group. The biotin group on the tether can now bind to streptavidin on the non-magnetic NP (i.e., reporter particle) forming a non-magnetic NP/MNP complex. Again, in this process, the MNP may be, for example, a 1.5-2 μg avidin/mg particle (50-60 molecules avidin/particle). Process 2100 may include, but is not limited to, the following steps.
(1) Magnetic bead preconcentration of the virus, wherein a MNP with anti-viral antibody thereon is used in a preconcentration process to capture and concentrate viral particles present in a sample;
(2) Viral lysis, wherein viral particles are lysed thereby allowing for detection of multiple viral targets per pathogenic particle;
(3) Isothermal amplification of viral nucleic acid sequences (e.g., using a loop-mediated-isothermal amplification (LAMP) or a reverse transcriptase-LAMP (RT-LAMP) protocol);
(4) Detection of virus, where in the presence of viral nucleic acid sequences the CRISPR enzyme will cleave the tether on the MNP thereby exposing the biotin tag for binding to streptavidin-functionalized non-magnetic NP forming a non-magnetic NP/MNP complex. A washing operation is performed and if virus was present, the non-magnetic NP remains anchored to the MNP via the streptavidin-biotin linkage and can be detected in a colorimetric reaction using HRP and TMB. If no virus was present, then no non-magnetic NP/MNP complex was formed and the non-magnetic NP bead is washed away and no color can be detected.

In another example of process 2100, the non-magnetic NP may be replaced with an AuNP and the presence of virus can be detected using the accumulation/depletion of color in a process similar to a lateral flow assay (e.g., as described above with reference to FIG. 15).

In yet another example of process 2100, any of the aforementioned processes 1400, 1500, 1600, 1700, 1800, 1900 may be applied to process 2100 as the read out for detecting the presence (or absence) of virus.

### EXAMPLES

### Example 1

### TMB⁺-Mediated Rapid Etching of Urchin-Like Gold Nanostructures for H₂O₂ Detection

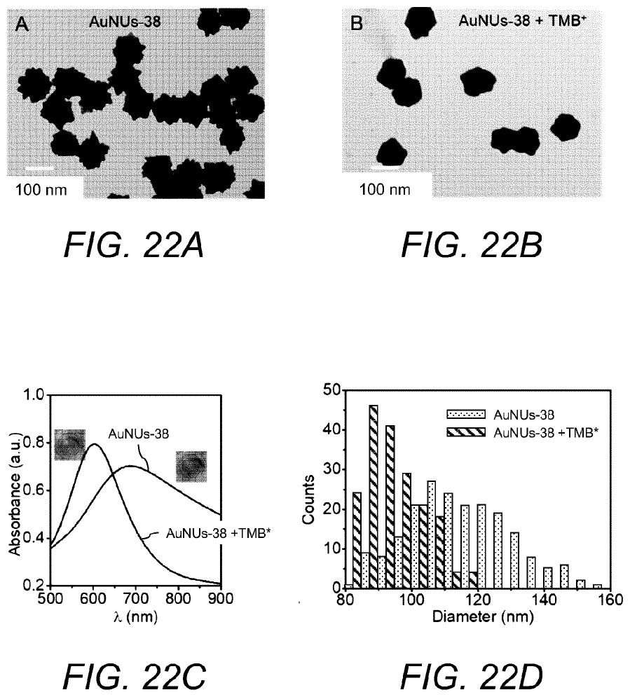

As shown and described hereinbelow, FIG. 22A through FIG. 36B show various plots, images, and diagrams supporting an example of the presently disclosed methods for signal enhancement and LOD improvement in plasmonic nanoparticle assisted ELISA in a fluidics device.

Hydrogen peroxide (H₂O₂) is an important molecule in biology. As a byproduct of many enzymatic reactions, H₂O₂ is a popular target of many sensors. A classic H₂O₂ sensor is based on the color change of 3,3′,5,5′-tetramethylbenzidine (TMB). In the presence of peroxidases (e.g., horseradish peroxidase HRP) and H₂O₂, the chromogenic TMB substrate can be oxidized to colored products TMB⁺. However, the sensitivities of these colorimetric detectors observed by naked eyes were limited by the final oxidized product concentrations. Gold nanoparticle-etching-based colorimetric sensors are powerful and highly sensitive. In previous work, the dye TMB²⁺ was found to etch gold nanorods (AuNRs) quantitatively and efficiently. Since the preparation of TMB²⁺ requires the extra addition of strong acid solution, AuNR-etching-based sensors always need multiple steps and acid conditions. As is described herein below, we developed a new colorimetric biosensing platform for H₂O₂ detection with urchin-like gold nanoparticles (AuNUs). Compared with AuNRs, the etching of AuNUs can happen under mild conditions in the presence of TMB⁺ at pH 6. Such mild reaction conditions also ensured the peroxidase activity of HRP during the AuNUs etching process. Therefore, the colorimetric detection of H₂O₂ by AuNUs can be realized by one-step mixing. In addition, the AuNUs-etching-based sensors were also fast (in 30 min) and sensitive. This system can sensitively detect H₂O₂ with a limit of 80 nm (2.7 parts-per-billion).

Gold nanoparticles (AuNPs) possess much higher extinction coefficients than organic dyes due to their localized surface plasmon resonance (LSPR), allowing visual observation at low nanomolar and even picomolar concentrations. The positions of LSPR peaks are dependent on the sizes and morphologies of AuNPs. Therefore, colorimetric detection can be made based on morphology changes of AuNPs, especially anisotropic AuNPs. Over the past decades, anisotropic AuNPs etching-based sensors are very prevalent. For example, the etching of AuNRs can happen along the longitudinal direction, leading to continuous color changes. However, a high concentration of cetrimonium bromide (CTAB) appeared essential for etching the AuNRs. In the presence of CTAB, the redox potential of AuBr²⁻/Au⁰ (0.93 V vs NHE) can be dramatically decreased by the AuBr²⁻-(CTA)²⁺/Au complex (<0.2 V vs NHE). The addition of hydrogen peroxide (H₂O₂), acids, and O₂ can also help the oxidation process of AuNRs. However, a high CTAB concentration (usually over 50 mm) and extreme conditions (acid or heating) limited the applications of AuNRs. Therefore, this example describes the exploration of gold nanostructures other than AuNRs that might be etched more easily under mild conditions.

Urchin-like AuNPs (AuNUs) are important anisotropic AuNPs in a broad range of applications from biosensors to cancer therapy. AuNUs can grow on AuNP seeds when additional Au³⁺ was reduced by sodium citrate and hydroquinone. The tip areas on the AuNUs are known to be highly reactive because of their high surface energy, and the sharp tips exhibit larger electric fields at their concavo-convex sites compared to neutral curvature areas. With these sharp tips, morphological changes of AuNUs can be triggered easily. For example, upon laser irradiation, the heat generated from the photothermal effect can melt the AuNUs into spherical AuNPs.

Enzyme-linked immunosorbent assay (ELISA) based detection systems have been widely used for the detection of various kinds of disease biomarkers. 3,3′,5,5′-tetramethylbenzidine (TMB) is an important substrate that can be oxidized to TMB⁺ (blue) or TMB²⁺ (yellow) for colorimetric immunoassays. It was reported that TMB²⁺ can quantitatively and efficiently etch AuNRs, which converted the color of TMB²⁺ to the color change of AuNRs to increase the sensitivity of detection. However, as mentioned above, acids were needed for this reaction to occur.

In this work, TMB⁺ induced etching of AuNUs was studied, and comparisons were made with AuNRs. In particular, the role of CTAB was investigated and its effect on the surfactant part was separated from its effect on the halide part. In the presence of a low concentration of CTA⁺ and Br ions, TMB⁺ can efficiently etch the branches of AuNUs. As a result, the morphology change of AuNUs was accompanied by a vivid color variation. Based on these understandings, we used the TMB⁺-induced etching of AuNUs and designed a colorimetric biosensing platform for H₂O₂ detection without any complex steps.

Materials and Methods

Chemicals. Gold (III) chloride trihydrate (HAuCl₄·3H₂O), H₂O₂ (30 wt %), 3,3′,5,5′-tetramethylbenzidine (TMB), sodium oleate (NaOL), Triton X-100, Tween 80, and Tween 20, and acetic acid were from Sigma-Aldrich (St Louis, MO). Cetrimonium chloride (CTAC), cetrimonium bromide (CTAB), sodium chloride, sodium fluoride, sodium bromide, sodium hydroxide, sodium citrate, sodium phosphate were from Mandel Scientific (Guelph, Ontario, Canada). Milli-Q water was used for preparing buffers and solutions.

Preparation of spherical Au seeds. Citrate-capped Au seeds were synthesized according to early reported literature. Briefly, the 100 mL 1 mm HAuCl₄ was heated and boiled for 30 s. Then, 38.8 mm sodium citrate was added quickly. The mixture solution will change from light yellow to wine red in 2 min. 13 nm AuNPs were obtained after refluxing for another 20 min. The same protocol was used for preparing 38 nm Au seeds. The concentration of HAuCl₄ was doubled to obtain 38 nm Au seeds.

Preparation of AuNUs. First, 30 mm hydroquinone solution was freshly prepared with Milli-Q water and used in one day. For a typical synthesis process, 1 mL HAuCl₄ was diluted with 180 mL H₂O under vigorous stirring. Subsequently, 600 μL Au seeds, 3 mL 38.8 mm sodium citrate, and 10 mL 30 mm hydroquinone was added one by one. 13 nm and 38 nm spherical Au seeds were, respectively, used for generating AuNUs-13 and AuNUs-38 NPs. The solutions were incubated at room temperature for 30 min under stirring. In the end, the AuNUs were washed with 5 mm pH 6 phosphate buffer at 4000 rpm for 8 min and stored at 4° C. for further use. The morphologies of AuNUs were characterized by TEM (Phillips CM10 100 kV) and UV-vis spectra.

Preparation of AuNRs. AuNRs were prepared by a seed-mediated method using a binary surfactant system as reported by C. B. Murray. For seed preparation, first, 5 mL 0.5 mm HAuCl₄ was added into 5 mL 0.2 M CTAB solution in a 20 mL scintillation vial. Second, 0.6 mL of ice-cold fresh 0.01 M NaBH₄ was diluted to 1 mL with water and was injected into the HAuCl₄-CTAB mixture under rapid stirring (1200 rpm). After stirring for 2 mins, the color of the solution changed from yellow to brown, and the seed solution was used after standing for min at room temperature. To prepare a growth solution, 7.0 g CTAB and 1.234 g NaOL were dissolved in 250 mL of warm water (50° C.) in an Erlenmeyer flask. After the solution was cooled to 30° C., 18 mL 4 mm AgNO₃ was added to the solution under stirring. The mixture was kept undisturbed at 30° C. for 15 min, then the 250 mL of 1 mm HAuCl₄ solution was added and stirred for 90 min. 1.5 mL HCl (37 wt % in water) was further added into the solution and stirred for 15 min. 1.25 mL of 0.064 M ascorbic acid (ΔA) was added into the solution under vigorously stirred for 30 s. Finally, 0.4 mL seed solution was injected into the growth solution with stirring for 30 s. The growth solution was left undisturbed for 12 hrs. Finally, the AuNRs suspension was centrifuged at 7000 rpm for 30 min to remove excess emulsifier, and washed once with water. The final AuNRs was stored in 250 mL 5 mm CTAB solution at 4° C.

Preparation of TMB⁺ and TMB²⁺. 2 mL 0.5 mm TMB substrate in 5 mm pH 4 buffer solution was irradiated under UV light (˜370 nm) for 30 min to get blue TMB⁺. The final concentration of TMB⁺ was determined by the absorbance of TMB⁺ at 652 nm with the extinction coefficient (ε=3.9 10⁴ M⁻¹cm⁻¹). TMB²⁺ was prepared by mixing TMB⁺ stock solution and 250 mm H₂SO₄ with a 1:1 volumetric ratio. The final concentration of TMB²⁺ was determined by the absorbance of TMB²⁺ at 450 nm with the extinction coefficient (ε=5.9 10⁴ M⁻¹cm⁻¹).

Etching of AuNUs by TMB⁺. In a typical etching experiment, 80.5 μL H₂O, 7.5 μL 100 mm CTAC, 30 μL 100 mm pH 6 phosphate buffer, 20 μL AuNUs, 3 μL 500 mm NaBr were added into microtubes in sequence. Then, 9 μL TMB⁺ with various concentrations was pipetted into microtubes respectively. The final volumes of samples were 150 μL. After vigorous stirring for 30 s, the samples were incubated at room temperature for 30 min before UV-vis absorbance measurements.

Colorimetric protocol for H₂O₂ detection. First, a mixture solution was prepared with TMB substrate, pH 6 phosphate buffer, and CTAC. Subsequently, 1 μL 0.1 mg/mL HRP was added into the mixture solution in the 96-well plates, followed by the addition of 20 μL AuNUs-13 NPs, 3 μL 500 mm NaBr, and various amounts of H₂O₂. The total volume in each well is 150 μL. The final concentrations of TMB substrate, phosphate buffer, and CTAC were respectively 100 μm, 20 mm, and 5 mm. Then, the mixture solution at room temperature for 30 min. Finally, the UV-vis absorbances were monitored by a plate reader, or the colors were distinguished by naked eyes.

TMB⁺-mediated etching of AuNUs. Referring now to FIG. 22A through FIG. 22D, FIG. 22A shows a TEM image of AuNUs-38 NPs before the addition of TMB⁺. FIG. 22B shows a TEM image of AuNUs-38 NPs after the addition of TMB⁺. FIG. 22C shows a plot of the UV-vis absorbance spectra of AuNUs-38 NPs before and after etching. Inset photos are Au corresponding samples. FIG. 22D shows a plot of the distribution histograms of the size change of AuNUs-38 NPs.

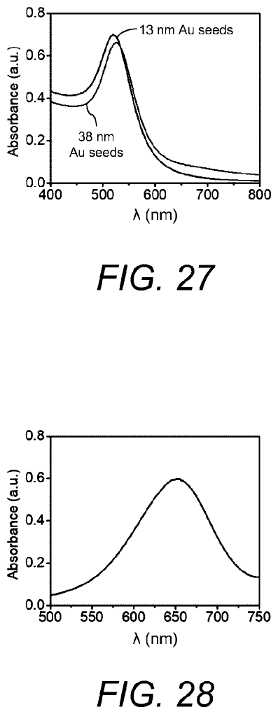

The AuNUs were synthesized by a seed-mediated growth method. Spherical AuNPs, which were prepared by citrate reduction, were used as seeds. Because the size of the AuNP seeds was around 38 nm, the resulting urchin-like products were called AuNUs-38. AuNUs grown from 13 nm seeds were also made and named AuNUs-13. From the TEM image shown in FIG. 22A, these AuNUs-38 had multiple sharp tips. Most of the AuNUs-38 were between 100 nm and 130 nm (see FIG. 22D). Compared with the spherical AuNP seeds with a surface plasmon resonance (SPR) peak at 526 nm (see FIG. 27), the SPR peak of AuNUs-38 showed a large red-shift, yielding a blue solution.

These sharp edges have higher surface energy, and they might be more easily etched. TMB is a common chromogenic substrate and upon one-electron oxidation, the TMB⁺ product has a blue color. An experiment was preformed to use TMB⁺ to etch the AuNUs to amplify the color change. To avoid potential effects of other molecules, TMB⁺ was produced by using UV irradiation (so no H₂O₂ or HPR was added). It needs to be noted that this method only yielded around 11% of TMB⁺, while the rest 89% was still the unreacted TMB substrate (see FIG. 28).

Then TMB⁺-mediated etching of the AuNUs was studied. In the presence of 5 mm CTAB, the sharp tips of AuNUs-38 were etched and rounded by the added TMB⁺ (see FIG. 22B). The blue color of the AuNUs-38 solution turned red in 30 min. Although the etched products were not in a spherical shape, a significant blue shift of SPR peaks (from 691 nm to 603 nm) was observed (see FIG. 22C). From the histogram shown in FIG. 22D, the size of etched AuNUs-38 NPs was also reduced from 110 nm to around 90 nm.

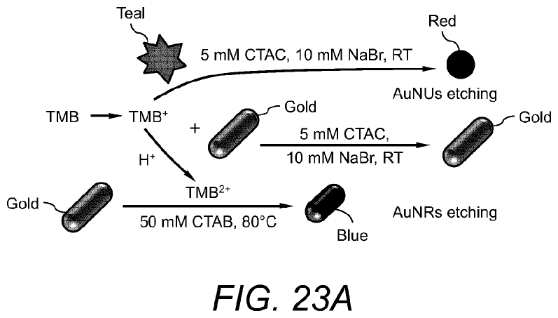

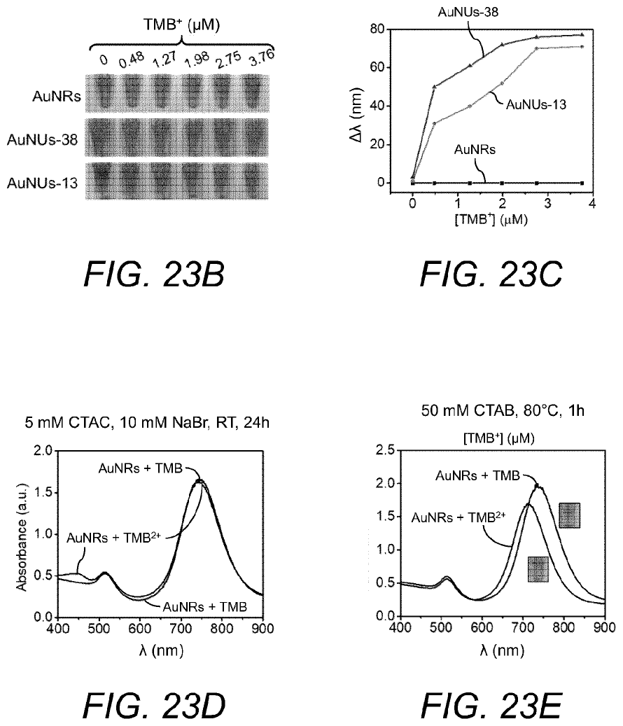

AuNUs are more easily etched than AuNRs. Referring now to FIG. 23A through FIG. 23E, FIG. 23A shows a schematic diagram of the etchings of AuNUs and AuNRs by TMB⁺ and TMB²⁺ respectively. FIG. 23B shows images in grayscale of changes of three AuNPs incubated with increasing concentration of TMB⁺ in which the AuNRs in the top row remained a consistent gold color, the AuNUs-38 in the middle row started out a greenish brown color at 0 TMB⁺ on the left and very gradually turned a greenish purple color by 2.75 and 3.76 μM TMB⁺, and the AuNUs-13 on the bottom row started out a bright teal color at 0 TMB⁺, turned a greenish purple color by 0.48 μM TMB⁺ and remained a light purple color from 1.27-3.76 μM TMB⁺. FIG. 23C shows a plot of SPR peak shifts of AuNRs, AuNUs-38, and AuNUs-13 after one-hour incubation with various TMB⁺ concentrations. In these etching experiments, 5 mm CTAC and 10 mm NaBr were used. Further, 10 μm TMB²⁺ was used in these experiments. FIG. 23D shows a plot of the absorption spectra measurements of AuNRs reacted with TMB²⁺ in the presence of CTAC. FIG. 23E shows a plot of the absorption spectra measurements of AuNRs reacted with TMB²⁺ in the presence of CTAB.

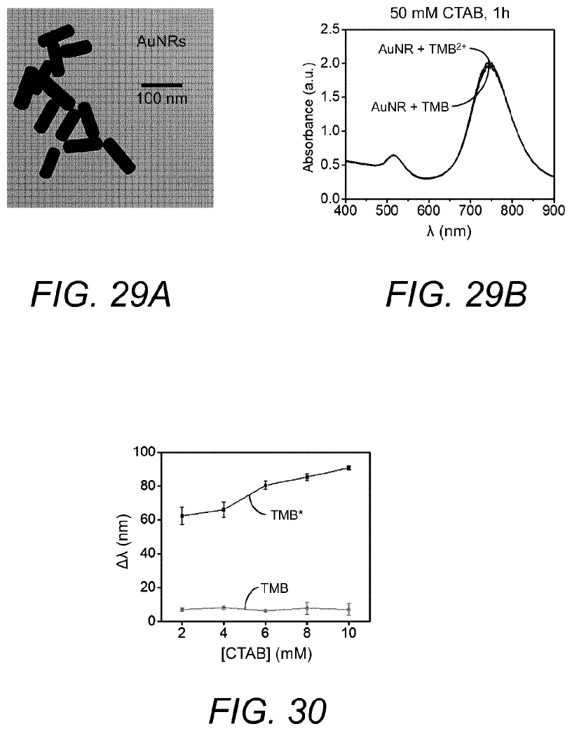

Since previous work mainly used AuNRs, the etching of AuNRs and AuNUs was compared. We expected that AuNUs could be more sensitive for TMB⁺ since the AuNRs lack the branches with high surface energy (see FIG. 29A). It was noticed that while AuNUs could be etched using TMB⁺, literature-reported etching of AuNRs used TMB²⁺ (see FIG. 23A). TMB²⁺ etched AuNRs along their longitudinal direction, leading to a continuous color change.

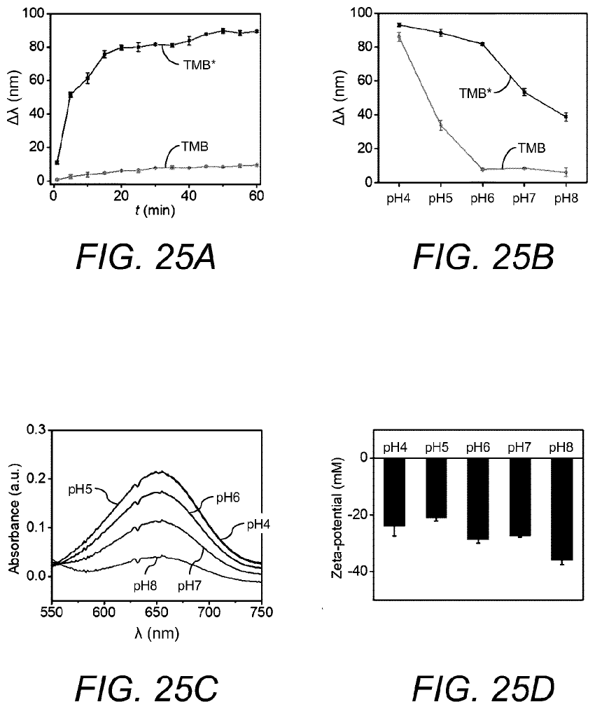

We also studied AuNUs-13 which are much smaller than AuNUs-38 in size, but with the same morphologies. All the three AuNPs were incubated with various TMB⁺ concentrations at room temperature. Interestingly, the AuNRs were quite stable under the etching conditions for AuNUs (see FIG. 23B, FIG. 23C). Even after 24-hour incubation, no absorbance peak shift happened for AuNRs (see FIG. 25D). To etch the AuNRs, a quite high CTAB concentration (50 mm), strong acids, TMB²⁺, and a high temperature of 80° C. were all required (see FIG. 29B). Even under such a harsh condition, a slight shift was observed in the absorption spectra, and no obvious color changes were observed in 1 h (see FIG. 23E).

For AuNUs, after one-hour incubation, color changes happened with both AuNUs-38 and AuNUs-13 as described above for FIG. 23B. Although these AuNUs with smaller sizes showed smaller SPR peak shifts, a much more obvious color change was obtained as can be observed in the change in wavelength shown in FIG. 23C. Therefore, for the subsequent studies, the larger AuNUs were used for quantitative spectroscopic measurements while the smaller AuNUs were used for sensing.

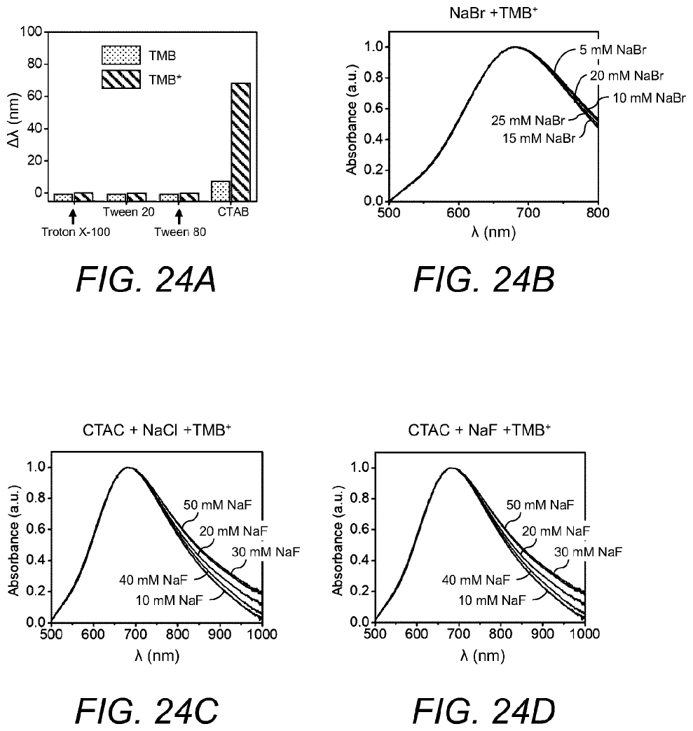

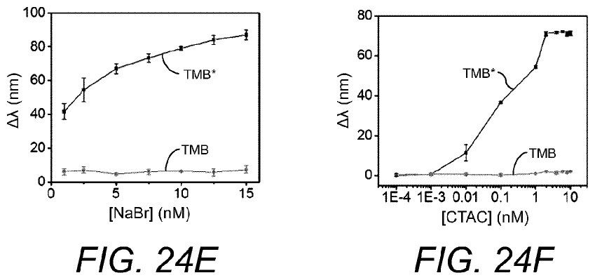

Effects of halides and surfactants. Referring now to FIG. 24A through FIG. 24F, FIG. 24A shows a plot of the SPR peak shifts of AuNUs-38 etched in the presence of 0.1% surfactants and wherein 0.1% CTAC & CTAB were respectively 3.1 mm and 2.7 mm. FIG. 24B shows a plot of the normalized absorption spectra of AuNUs incubated with the mixture of various NaBr concentrations and 4 μm TMB⁺. FIG. 24C shows a plot of the effects of F⁻ (chloride) on the etching of AuNUs-38 in the presence of 5 mm CTAC and 4 μm TMB⁺. FIG. 24D shows a plot of the effects of Cl⁻ (fluoride ions) on the etching of AuNUs-38 in the presence of 5 mm CTAC and 4 μm TMB⁺. (F) SPR peak shifts of AuNUs-38 NPs as a function of NaBr. In these experiments, 5 mm CTAC was used. SPR peak shifts of AuNUs-38 etched by various concentrations of CTAC and fixed 10 mm NaBr.

After demonstrating the feasibility, to acquire the best etching performance, the effects from the reaction conditions, such as pH, etching time, surfactant, and halide ion were investigated in detail. The difference in SPR peak positions (Δλ) was chosen as the indicator of the morphological change of the AuNUs.

The experiment was started by optimizing surfactants. The as-synthesized AuNUs were mainly covered by weakly adsorbed citrate groups, which can be easily displaced by other stronger capping agents. During the initial experiments, cetrimonium bromide (CTAB) was used as the capping agent since CTA⁺ has been well studied on AuNRs etching. Herein, a few common surfactants were also evaluated, and interestingly, TMB⁺ only etched the AuNUs in the presence of CTAB (see FIG. 24A). It was also noticed that the etching was faster with higher CTAB concentrations (see FIG. 30).

CTAB contained Br as its counterion, and Br has a strong affinity to the gold surface. Halides counterions of the surfactant have been proved to be critical in the oxidation of CTAB-capped AuNRs. Halides adsorption was widely studied for shape-controlled AuNP synthesis. It is known the adsorbed concentrations of Br⁻ and I⁻ are much higher than that of Cl⁻ ions on Au surface, and the interaction strength of halides with gold has the trend of I⁻>Br>Cl⁻. Thus, it was expected that Br might be important during the etching process. Interestingly, without CTA⁺, only Br failed to induce the etching process (see FIG. 24B). Therefore, CTA⁺ appeared to be required for AuNUs etching.

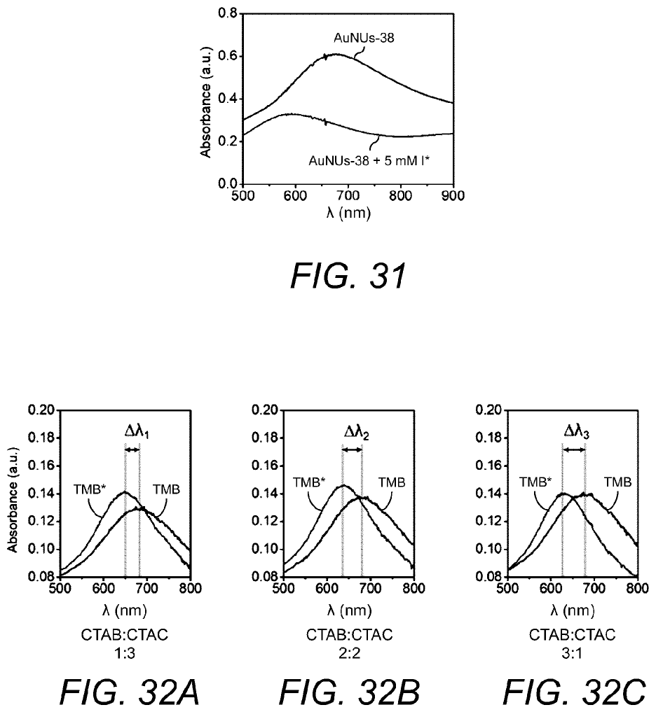

To confirm the roles of halide counterions for deeper mechanistic understanding, more etching experiments were conducted with the mixture of cetrimonium chloride (CTAC) and three halides (F⁻, Cl⁻, and Br⁻). I⁻ was omitted here because a low concentration of iodide can cause serious etching of AuNUs without the help of CTA⁺ groups (see FIG. 31). With 5 mm CTAC and up to 50 mm F⁻ or Cl⁻, no SPR peak shifts were observed with the incubation with 4 μm TMB⁺ for 30 min (see FIG. 24C, FIG. 24D). These trends are consistent with the etching of AuNR and Au nanostar. Therefore, Br and CAT were both essential for etching the AuNUs. The importance of Br was also confirmed by conducting etching experiments in the mixture of 0.25 mm CTAB 0.75 mm CTAC, 0.5 mm CTAB 0.5 mm CTAC, and 0.75 mm CTAB 0.25 mm CTAC (see FIG. 32). Greater Δλ was generated with a higher portion of CTAB, again indicating the involvement of Br in the etching process.

To get fast etching speeds or large SPR peak shifts, higher CTAB concentrations are better. However, a too high CTAB concentration may produce many bubbles. These bubbles can cause problems for quantitative absorbance measurements. Also, CTAB has poor solubility at room temperature. Therefore, it was necessary to achieve fast etching with the lowest possible surfactant concentration. This goal is possible by using a mixture of CTAC and NaBr.

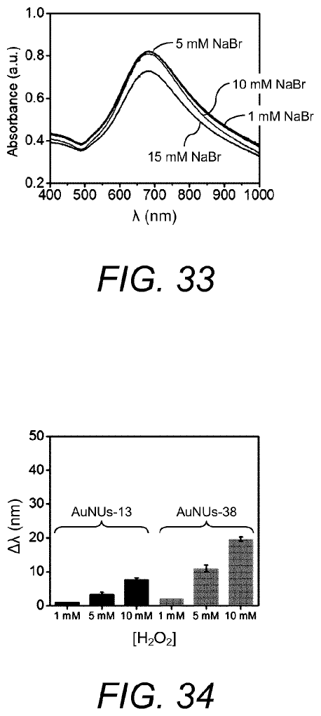

The CTAC concentration was fixed at 5 mm (10-folder lower than the typical 50 mm CTAB concentration), and larger SPR peak shifts happened with more NaBr added (see FIG. 24E). To avoid the aggregation of AuNUs, 10 mm NaBr concentration was chosen for the following work (see FIG. 33). With fixed 10 mm NaBr, larger SPR peak shifts were observed with higher CTAC concentrations (see FIG. 24F). When CTAC concentration was higher than 2 mm, negligible improvements were obtained. This can be explained by the fact that the critical micelle concentrations (CMC) of CTAB and CTAC are both around 1 mm at room temperature.^{31 32} Therefore, it was chosen to use 5 mm CTAC in this work. In addition, no surfactant precipitation was observed with 5 mm CTAC and 10 mm NaBr during the etching process.

Optimization of etching time and pH. Referring now to FIG. 25A through FIG. 25D, FIG. 25A shows a plot of the etching kinetics of AuNUs-38 where SPR peak shifts were used. FIG. 25B shows a plot of the SPR peak shifts of AuNUs-38 which were incubated in different pH environments. Acetate buffers were used for pH 4 & 5; phosphate buffers were used for pH 6-8. FIG. 25C shows a plot of the stabilities of TMB⁺ produced by UV light at different pHs. FIG. 25D shows a plot of the zeta-potentials of AuNUs-38 at different pHs.

After understanding the effects of surfactants and Br, the etching time and pH conditions were further investigated. FIG. 25A shows that the etching happened fast in 20 min, after which the shift of SPR peak was pretty slow. For the control sample (TMB substrate), only a minor increase in Δλ (9 nm) after even 1 h. As a result, all the etching samples were incubated for 30 min to get a large and relatively stable difference in Δλ.

Then the effects of pH on the etching were studied. An acidic environment was shown to facilitate Au nanomaterial etching in the presence of dissolved oxygen. However, many proteins are only stable in a narrow pH range near neutral. For example, horseradish peroxidase (HRP) loses its structural and conformational stability at pH<4. Thus, the etching of AuNUs-38 was studied between pH 4 and 8. From FIG. 25B, at pH 4, the Δλ of the TMB control sample was very close to TMB⁺. The color difference of these two samples was not distinguishable with such close absorbance peak positions. As such, the etching was mainly caused by the low pH, and detection of TMB⁺ would be impossible at pH 4. The largest Δλ difference was observed at pH 6. When the pH is higher than 6, the etching activity of TMB⁺ decreased dramatically. This can be explained by the low stability of TMB⁺ at higher pH (see FIG. 25C). Therefore, pH 6 was chosen as the optimum pH for further study. In terms of surface charges of AuNUs-38, all of them were negatively charged from pH 4 to 8 and thus should not be the reason for the difference (see FIG. 25D).

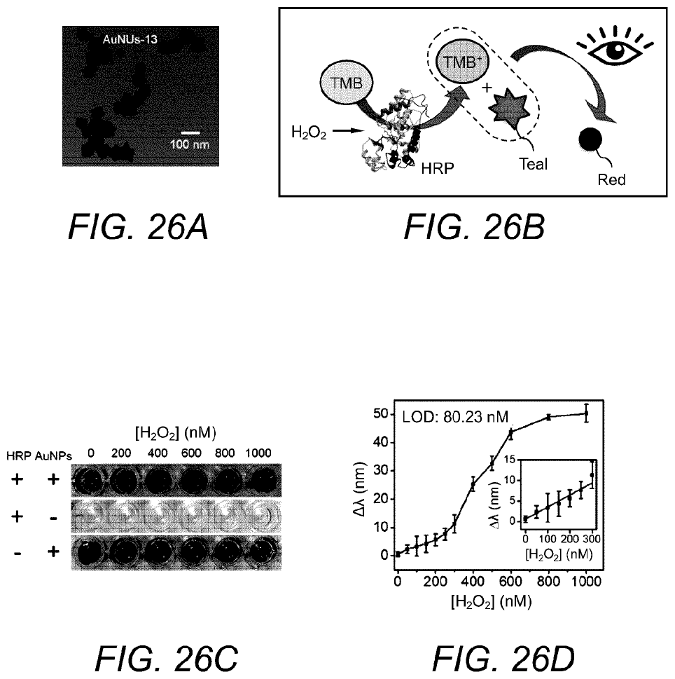

Visual detection of H₂O₂. Referring now to FIG. 26A through FIG. 26D, FIG. 26A shows a TEM image of AuNUs synthesized from 13 nm spherical AuNPs. FIG. 26B shows a schematic diagram of the etching of AuNUs induced by the product of HRP-catalysed TMB. FIG. 26C are images in grayscale of the color changes of the AuNPs used in the described method with the increase of H₂O₂ concentration. In FIG. 26C, the top row wells are a bluish green color at 0 and 200 nM H₂O₂, a purplish color at 400 nM, and a reddish purple at 600 nM and greater concentration of H₂O₂. In FIG. 26C, the middle row wells are consistently transparent and colorless. In FIG. 26C, the bottom row wells are consistently a bluish green color like the 0 and 200 nM H₂O₂ wells of the top row. FIG. 26D shows a plot of the LSPR shifts of AuNUs-13 as a function of H₂O₂ concentration. Inset: the response at a low concentration range.

H₂O₂ is an important by-product of many enzymatic reactions and has been used as a target molecule of many biosensors. For example, the oxidation of glucose by glucose oxidase (GOx) can produce H₂O₂. It is well known that H₂O₂ can oxidize AuNRs in the presence of Br under acid conditions at high temperatures. However, the concentration used for AuNRs oxidation is much higher than that used in the experiments described herein. Over 1 mm H₂O₂ is required to cause slight etching of AuNUs (see FIG. 34).

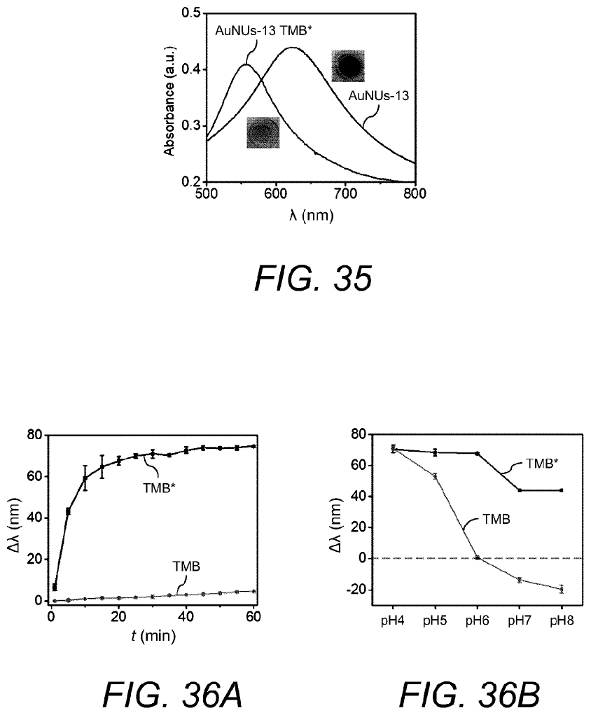

To realize the visual detection, the AuNUs synthesized from smaller Au seeds were used. A more vivid color change was generated with the sharp tips characteristic but smaller as described above for FIG. 23B and shown in FIG. 26A. The experimental setup is similar to the conventional colorimetric ELISA. Horseradish peroxidase (HRP) enzymes can catalyze H₂O₂ to produce a more reactive radical species (OH). Then OH was quantitatively reacted with TMB substrate to generate TMB⁺ (see FIG. 26B). Thanks to the mild conditions for AuNUs etching, HRP-H₂O₂ catalysis and AuNUs etching can be realized in one step. After TMB⁺-mediated etching, an obvious blue shift for the SPR peak happed (see FIG. 35). The etching reaction also happened fast in 20 min, and the control samples are very stable at pH 6 (see FIG. 36). Only the samples with HRP displayed a color change (from blue to red). The images of color change are described above for FIG. 26C.

This is one of the most sensitive colorimetric sensors, which showed significant color change when the concentration of H₂O₂ is equal to or higher than 400 nm. Under conditions used in this study, the limit of detection for H₂O₂ is 80.23 nm (3σ/slope, inset) (see FIG. 26D). This is one of the most sensitive AuNP-based colorimetric sensors for H₂O₂ detection (e.g., ˜7-fold more sensitive than the previous colorimetric detection).

In summary, a new one-step colorimetric biosensing platform for H₂O₂ detection by TMB⁺-mediated etching of AuNUs has been successfully developed. All CTA⁺, TMB⁺, and Br molecules were important in etching AuNUs. The reaction conditions including time, pH, and surface ligands were also optimized. Although the blue color of TMB⁺ with low concentrations failed to be discerned with the naked eyes, nanomolar level TMB⁺ can still cause vivid color changing of AuNUs solution by etching. This work demonstrates a high sensitivity sensor for H₂O₂ and also expands TMB⁺-mediated etching of gold nanomaterials.

### Example 2

### Camera Detection of Gold Nanoparticle Etching by Oxidized TMB on Digital Microfluidic Device

Reagent Preparation. Reagent 1 was prepared by mixing citrate coated gold nanourchins suspended in deionized water (18 Mohm) with cetrimonium chloride (CTAC). The final concentration of gold nanourchins and CTAC were 10 OD and 4 mM, respectively.

Commercial 3,3′,5,5′-Tetramethylbenzidine (TMB) was mixed with biotin-HRP to prepare oxidized TMB. The concentration of oxidized TMB was measured with a UV-Vis spectrophotometer.

Gold Nanourchin Etching on Digital Microfluidic (DMF) Cartridge. All reagents listed in Table 1 were loaded independently onto the DMF cartridge. The reagents were dispensed and transported as aqueous droplets through the oil filled DMF cartridge to specific locations on the cartridge, where reagent mixing took place.

Eight samples were prepared on cartridge using various combinations of Reagents 1-3 (Table 1). Each sample consists of two droplets of Reagent 1, one droplet of Reagent 2, and one droplet of Reagent 3. First, Reagent 1 & 2 were mixed and held as a stationary droplet for a few minutes. During this period, images were taken with the Blackfly S USB3 camera (Model bfs-u3-200s6c, Data not shown). Then, Reagent 3 was mixed with the combined Reagent 1&2 droplet. The combined droplet was then held as a stationary droplet. More images were taken with the Blackfly S USB3 camera Data not shown.

Image Analysis. The HSV color scale was used for measuring the colorimetric change of the samples and the average hue values of the samples were measured using GIMP (Table 2). During image analysis, areas of the droplet that were severely impacted by lighting, shadows or bubbles were excluded from hue measurements.

Results. After mixing Reagent 1 and Reagent 2, all samples appeared blue to the eye and had an average hue between 223.5 to 230.0. After mixing with Reagent 3, the samples that received 4 uM oxidized TMB appeared to be more purple to the eye compared to the other samples and had a higher average hue (see Table 3). The greater change in hue among these samples is due to etching of the gold nanourchins by oxidized TMB.

TABLE 1
Sample Composition

| Sample | Sample | | | |
|---|---|---|---|---|
| Group | ID | Reagent 1 | Reagent 2 | Reagent 3 |
| A | 9 | 10 OD gold | 160 mM sodium | Deionized water + |
| | 13 | nanourchins + 4 mM | bromide + 0.1% | 0.1% Tween20 |
| | | CTAC | Tween20 | (weight/volume) |
| | | | (weight/volume) | |
| B | 10 | 10 OD gold | 160 mM sodium | TMB + 0.1% |
| | 12 | nanourchins + 4 mM | bromide + 0.1% | Tween20 |
| | 15 | CTAC | Tween20 | (weight/volume) |
| | | | (weight/volume) | |
| C | 11 | 10 OD gold | 160 mM sodium | 4 uM Oxidized TMB + |
| | 14 | nanourchins + 4 mM | bromide + 0.1% | 0.1% Tween20 |
| | 16 | CTAC | Tween20 | (weight/volume) |
| | | | (weight/volume) | |

TABLE 2
Average Hue Value of Each Sample at Different Timepoints

| Time | | | | | | | | | |
|---|---|---|---|---|---|---|---|---|---|
| After | | | | | | | | | |
| Mixing | | | | | | | | | |
| Reagents | | | | | | | | | |
| 1 & 2 | | | | | | | | | |
| With | Sample | Group A | | Group B | | | Group C | | |
| Reagent 3 | ID | 9 | 13 | 10 | 12 | 15 | 11 | 14 | 16 |

| | | | | | | | | |
|---|---|---|---|---|---|---|---|---|
| Reagent 1 + 2 Only | 230.0 | 225.5 | 227.0 | 224.5 | 226.0 | 225.0 | 225.0 | 223.5 |
| (Before Mixing | | | | | | | | |
| with Reagent 3) | | | | | | | | |
| 1 Minute | 224.5 | 224.0 | 222.0 | 224.0 | 224.0 | 227.0 | 226.5 | 224.0 |
| 8 Minutes | 225.5 | 226.5 | 227.0 | 227.5 | 233.0 | 249.0 | 249.0 | 254.0 |

TABLE 3
Average Change in Hue Value After Mixing Reagents 1 & 2 With Reagent 3

| Time | | | | | | | | | |
|---|---|---|---|---|---|---|---|---|---|
| After Mixing | | | | | | | | | |
| Reagents | | | | | | | | | |
| 1 & 2 With | Sample | Group A | | Group B | | | Group C | | |
| Reagent 3 | ID | 9 | 13 | 10 | 12 | 15 | 11 | 14 | 16 |

| | | | | | | | | |
|---|---|---|---|---|---|---|---|---|
| 1 Minute | −5.5 | −1.5 | −5.0 | −0.5 | −2.0 | 2.0 | 1.5 | 0.5 |
| 8 Minutes | −4.5 | 1.0 | 0.0 | 3.0 | 7.0 | 24.0 | 24.0 | 30.5 |

### Example 3

### Detection of Gold Nanourchin Etching on DMF after Substrate Oxidation by HRP-Beads

Reagent Preparation. Streptavidin coated magnetic beads were conjugated with biotin-HRP and suspended in buffer. Citrate coated gold nanourchins suspended in deionized water (18 Mohm) were mixed with cetrimonium bromide (CTAB) such that the final concentration of gold nanourchins and CTAB were 7 OD and 30 mM, respectively.

TMB Oxidation & Gold Nanourchin Etching on DMF Cartridge. Streptavidin coated magnetic beads, Biotin-HRP conjugated magnetic beads, gold nanourchins (10 OD) mixed with CTAB (30 mM), and TMB substrate were independently loaded onto the DMF cartridge. Each reagent was loaded into different wells on the cartridge for dispensing. All reagents were transported through the oil filled cartridge as aqueous droplets.

Streptavidin coated magnetic beads & biotin-HRP conjugated magnetic beads were magnetically separated from the original buffer solution on the DMF cartridge, and then resuspended in TMB. The TMB was allowed to incubate with magnetic beads for 15 minutes. After the 15 minute incubation period, the magnetic beads were magnetically separated from the TMB solutions. The TMB solutions mixed with deionized water at a 1:1 volume ratio on the cartridge.

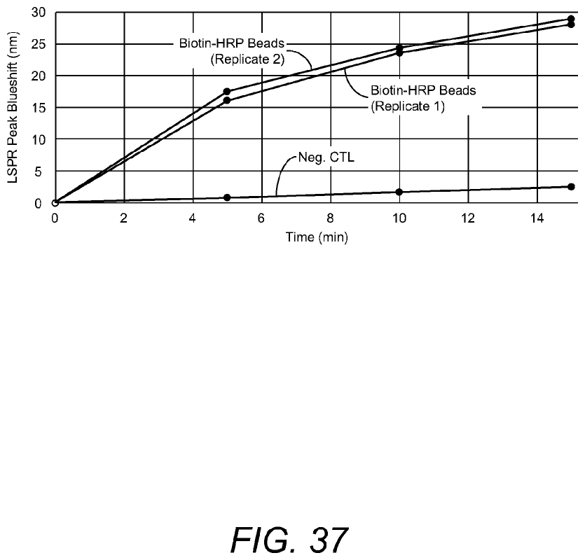

The diluted TMB solutions were then mixed with gold nanourchins (at a 1:1 volume ratio). The maximum LSPR peak position of the gold nanourchins was tracked over time with optical fibers immersed in the gold nanourchins+CTAB+TMB solution (see FIG. 37). The optical fibers illuminated the droplets (containing gold nanourchins) and collected scattered light from the droplets as input to the spectrophotometer.

Gold nanourchins that received TMB incubated with streptavidin coated magnetic beads are labelled as the negative control (NEG.CTL) in FIG. 37.

Results. The maximum LSPR peak position of gold nanourchins mixed with TMB incubated by biotin-HRP conjugated beads blue-shifted significantly more than the NEG.CTL. This result shows that biotin-HRP conjugated magnetic beads can oxidize TMB substrate on a DMF cartridge, and the oxidization of TMB can be detected by gold nanourchin etching, which results in a colorimetric change that can be measured by tracking the maximum LSPR peak position of the gold nanourchins.

### Example 4

### Detection of Spike Protein in a Plasmonics Assisted ELISA on a Digital Microfluidic Device

Reagent Preparation. Citrate coated gold nanourchins suspended in deionized water (18 Mohm) were mixed with cetrimonium bromide (CTAB) such that the final concentration of gold nanourchins and CTAB were 5.1 OD and 24 mM, respectively. R001-MNPs were prepared by conjugating magnetic beads with anti-spike antibody (otherwise referred to herein as “capture biomolecule”).

Spike Protein (S1 Subunit) Plasmonic ELISA on DMF. The following reagents were independently loaded into separate wells on the DMF cartridge: citrate coated gold nanourchins (7 OD) mixed with CTAB (30 mM), R001-MNPs, anti-spike HRP (D001-HRP; “secondary antibody” or “antibody” having conjugated “enzyme”), TMB substrate, deionized water, wash buffer (phosphate buffered saline+1% bovine serum albumin), spike protein (S1 subunit; “target analyte”) and phosphate buffered saline.

R001-MNPs were incubated with 250 μM spike protein (or phosphate buffered saline in the case of the negative control). The Spike-R001-MNPs (and R001-MNPs) were washed several times with wash buffer (by magnetic separation and resuspension). The magnetic beads were then incubated with secondary antibody, anti-spike HRP (D001-HRP). The magnetic beads were washed several times with wash buffer (by magnetic separation and resuspension). Next, the magnetic beads were suspended in TMB substrate and allowed to incubate for 15 minutes.

After the 15 minute incubation period, the magnetic beads were magnetically separated from the TMB solutions. The TMB solutions mixed with deionized water at a 1:1 volume ratio. The diluted TMB solutions were then mixed with gold nanourchins (at a 1:1 volume ratio). The maximum LSPR peak position of the gold nanourchins was tracked over time with optical fibers immersed in the gold nanourchins+CTAB+TMB solution (see FIG. 38). The optical fibers illuminated the droplets (containing gold nanourchins) and collected scattered light from the droplets as input to the spectrophotometer.

Gold nanourchins that received TMB incubated with magnetic beads that mixed with buffer instead of 250 pM spike protein are labelled as the negative control (NEG.CTL) in FIG. 38.

Results. The maximum LSPR peak position of gold nanourchins mixed with TMB incubated by beads that mixed with 250 pM spike protein blue-shifted significantly more than the NEG.CTL (FIG. 38). This result shows that spike protein in solution can be detected by performing plasmonic ELISA on DMF. The spike protein plasmonic ELISA consists of capturing the spike protein (target analyte”) with anti-spike antibody conjugated magnetic beads (capture biomolecule), labelling the bead-spike protein complex with secondary anti-spike HRP (antibody having conjugated enzyme), incubating the beads with TMB (substrate), followed by etching of gold nanourchins by the oxidized TMB, which results in a colorimetric change that can be measured by tracking the maximum LSPR peak position of the gold nanourchins.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “includes,” and “including” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Unless specifically stated otherwise, terms such as “receiving,” “routing,” “updating,” “providing,” or the like, refer to actions and processes performed or implemented by computing devices that manipulates and transforms data represented as physical (electronic) quantities within the computing device's registers and memories into other data similarly represented as physical quantities within the computing device memories or registers or other such information storage, transmission or display devices. Also, the terms “first,” “second,” “third,” “fourth,” etc., as used herein are meant as labels to distinguish among different elements and may not necessarily have an ordinal meaning according to their numerical designation.

Examples described herein also relate to an apparatus for performing the operations described herein. This apparatus may be specially constructed for the required purposes, or it may comprise a general purpose computing device selectively programmed by a computer program stored in the computing device. Such a computer program may be stored in a computer-readable non-transitory storage medium.

The methods and illustrative examples described herein are not inherently related to any particular computer or other apparatus. Various general purpose systems may be used in accordance with the teachings described herein, or it may prove convenient to construct more specialized apparatus to perform the required method steps. The required structure for a variety of these systems will appear as set forth in the description above.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. 112, sixth paragraph, for that unit/circuit/component. Additionally, “configured to” or “configurable to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A plasmonic particle assisted method of detecting a target analyte, comprising:
a. introducing a sample fluid potentially comprising a target analyte to a capture biomolecule in a microfluidic device, wherein binding of the target analyte to the capture biomolecule forms a target-capture biomolecule complex, and wherein, optionally, one or both of the capture biomolecule and a plasmonic particle is immobilized on a surface;
b. introducing an antibody that binds directly or indirectly to the target analyte at a different site than the capture biomolecule, wherein:
(i) an enzyme is conjugated directly or indirectly to the antibody, or
(ii) the enzyme is conjugated to the capture biomolecule;
c. introducing a substrate for the enzyme;
d. introducing a plasmonic particle;
e. measuring an optically detectable change caused by one or a combination of etching, growth, aggregation, or altered interparticle distance of the plasmonic particle in the vicinity of the target-capture biomolecule complex, wherein the etching, growth, aggregation, or altered interparticle distance is in response to a product or byproduct generated by a reaction between the enzyme and the substrate, wherein the amount of enzyme-substrate reactions is proportional to the number of target analytes bound to the capture biomolecules; and
f. optionally, quantifying an amount of the target analyte present in the sample based on the optically detectable change.

2. The method of claim 1, wherein the surface is a magnetic bead.

3. The method of claim 1 or 2, wherein the capture biomolecule is immobilized on the surface or on the magnetic bead and the plasmonic particle is introduced in a fluidic suspension subsequent to introduction of the substrate.

4. The method of claim 1, wherein the plasmonic particles are introduced as a fluidic suspension in a droplet in conjunction with the substrate or wherein the plasmonic particles are introduced as a fluidic suspension in a droplet separately from the substrate.

5. The method of claim 1, wherein the capture biomolecule and the plasmonic particle are both immobilized on the surface.

6. The method of claim 1, wherein the capture biomolecule is immobilized on the surface and the plasmonic particle is immobilized on a separate surface.

7. The method of any of the preceding claims, wherein measuring the optically detectable change is performed by one or both the naked eye, with an instrument, or with a camera.

8. The method of any of the preceding claims, wherein the sample fluid comprises a bodily fluid from a human or an animal.

9. The method of any of the preceding claims, wherein the target analyte is a protein, an antigen, an antibody, an IgG antibody, an IgM antibody, a virus, a molecule or molecular structure from a virus, a bacteria, or any other pathogen, or a molecule or molecular structure bound to the outer surface of a virus, a bacteria, or any other pathogen.

10. The method of claim 9, wherein the internal molecule or molecular structure is exposed by disrupting the integrity of the virus, the bacteria, or any other pathogen.

11. The method of any of claims 1-8, wherein the target analyte is a target antibody and wherein the capture biomolecule is an antigen that the target antibody binds to.

12. The method of any of claims 1-8, wherein the target analyte is a virus and the capture biomolecule is an antibody that binds to the virus.

13. The method of any of the preceding claims, wherein the optically detectable change is a colorimetric change

14. The method of any of the preceding claims, wherein the capture biomolecule is immobilized on the surface, the surface is a magnetic bead, and the antibody is conjugated indirectly to the enzyme as a result of both the antibody and the enzyme being conjugated to a second surface, the second surface is a non-magnetic bead.

15. The method of any of claims 1-13, wherein the capture molecule is immobilized on the surface, the surface is a magnetic bead, and the antibody is immobilized on a second surface.

16. The method of any of the preceding claims, wherein the enzyme comprises horseradish peroxidase (HRP), the substrate comprises TMB, and the optically detectable change comprises etching of the plasmonic particles by the oxidized TMB substrate.

17. The method of any of claims 1-13, wherein the enzyme comprises alkaline phosphatase (AP).

18. The method of any of the preceding claims, wherein the plasmonic particle comprises more than one layer, more than one material, or combinations thereof.

19. The method of claim 18, wherein the plasmonic particle comprises one or a combination of a plasmonic layer, a dielectric layer, a semiconductor layer, or a polymeric layer.

20. The method of claim 19, wherein the semiconductor layer comprises a magnetic, a paramagnetic, or a superparamagnetic semiconductor layer.

21. The method of any of the preceding claims, wherein the plasmonic particle comprises one or more particle dimensions of less than about 100 nm.

22. The method of any of the preceding claims, wherein the plasmonic particle is a core-shell particle, a rattle-type particle, a hollow particle, a porous particle, a bimetallic particle, a single metal particle, a gold nanorod, or a gold nanourchin.

23. The method of claim 22, wherein the core-shell particle comprises a gold core and an outer porous (or mesoporous) metal oxide layer, thereby facilitating enzyme catalyzed etching of the particle core for optical detection of the target analyte.

24. The method of claim 22, wherein the core-shell particle comprises a silver core and an outer porous (or mesoporous) metal oxide layer, thereby facilitating enzyme catalyzed etching of the particle core for optical detection of the target analyte.

25. The method of claim 23 or 24, wherein the metal oxide layer comprises silicon dioxide (SiO2@Ag or SiO2@Au).

26. The method of any of the preceding claims, further comprising introducing a fluorescent probe.

27. The method of claim 26, wherein the fluorescent probe is adhered or chemically bound to the plasmonic particle.

28. The method of claim 27, wherein the fluorescent probe comprises a quantum dot.

29. The method of claim 26 or 27, wherein the optically detectable change is caused by etching of the plasmonic particles which allows the fluorescent probe or quantum dot to be released or detached from the plasmonic particles.

30. The method of any of claims 26-29, further comprising introducing a fluorescence quencher/acceptor moiety.

31. The method of claim 30, wherein the fluorescence quencher/acceptor moiety comprises a black hole quencher.

32. The method of claim 30, wherein the fluorescence quencher/acceptor moiety comprises a short oligomer dual-labeled with a FRET pair.

33. The method of claim 32, wherein the short oligomer comprises a peptide, an aptamer, or a carbohydrate-based molecule.

34. The method of any of the preceding claims, wherein the plasmonic particle comprises two or more types of plasmonic particles, thereby increasing one or both of a sensitivity or a range of detection for the target analyte.

35. The method of any of claims 1-33, wherein the plasmonic particle comprises a mixture of one or more aggregation states of the same plasmonic particle, thereby increasing the sensitivity and/or range of detection for the target analyte.

36. The method of any of claims 1-33, wherein the plasmonic particle comprises a mixture of one or more aggregation states of one or more types of plasmonic particle, thereby increasing the sensitivity and/or range of detection for the target analyte.

37. The method of any of the preceding claims, wherein the optically detectable change is a colorimetric change, and further comprising using a background color wherein the background color is not changed in response to the enzyme-substrate reactions, thereby providing a constant measurable color.

38. The method of claim 37, wherein the background color is provided by a dye.

39. The method of claim 37, wherein the background color is provided by a second plasmonic particle that is not changed in response to the enzyme-substrate reactions.

40. The method of claim 39, wherein the second plasmonic particle comprises a protective surface modification, thereby providing resistance to a colorimetric change in response to the enzyme-substrate reactions.

41. The method of any of any of the preceding claims, wherein the reduced interparticle distance is mediated by an addition or a presence of ionic molecules containing one or more functional groups that can covalently bind to the particles, the addition or the presence of the ionic molecules a result of the enzyme-substrate reactions.

42. The method of claim 41, wherein the one or more functional groups comprise one or a combination of a carboxyl group, a thiol group, or an amine group.

43. The method of claim 41, wherein the one or more functional groups are present on the plasmonic particles, and wherein in response to the enzyme-substrate reactions a chemical bond is formed between the plasmonic particles, thereby aggregating or reducing the interparticle distance of the plasmonic particles.

44. The method of claim 43, wherein the chemical bonding of the plasmonic particles is provided by one or more different functional groups used in a click chemistry reaction.

45. The method of claim 44, wherein the click chemistry reaction comprises a thiol-ene reaction or a copper(I)-catalyzed azide-alkyne cycloaddition.

46. The method of any of claims 1-12, wherein the plasmonic particle comprises a core-shell particle and further comprising introducing a metal ion precursor, wherein the optically detectable change is caused by growth of the plasmonic particle in response to a reduction of the metal ion precursor mediated by the enzyme-substrate reactions.

47. The method of claim 46, wherein the core-shell particle comprises a thin shell formed in response to the enzyme-substrate reactions on top of a plasmonic core.

48. The method of claim 46, wherein the plasmonic core and the thin shell are the same metallic material.

49. A method of detecting a target analyte, comprising:
a. introducing a sample fluid potentially comprising a target analyte to a capture biomolecule in a microfluidic device, wherein binding of the target analyte to the capture biomolecule forms a target-capture biomolecule complex, and wherein, optionally, one or both of the capture biomolecule and a plasmonic particle is immobilized on a surface;
b. introducing an antibody that binds directly or indirectly to the target analyte at a different site than the capture biomolecule, wherein:
(i) an enzyme is conjugated directly or indirectly to the antibody, or
(ii) the enzyme is conjugated to the capture biomolecule;
c. introducing a substrate for the enzyme and a metal ion precursor, wherein the introducing is either at the same time or at separate times;
d. measuring an optically detectable change caused by nucleation and growth of a plasmonic particle in the vicinity of the target-capture biomolecule complex, wherein the optically detectable change is in response to a reduction in the metal ion precursor as a result of the nucleation and growth of the plasmonic particle mediated by a reaction between the enzyme and the substrate, wherein the amount of enzyme-substrate reactions is proportional to the number of target analytes bound to the capture biomolecules; and
e. optionally, quantifying an amount of the target analyte present in the sample based on the optically detectable change.

50. A system for performing plasmonic particle assisted detection of a target analyte, the system comprising:
a. a digital microfluidic (DMF) cartridge configured for plasmonic particle assisted enzyme-linked immunosorbent assay (ELISA), the DMF cartridge comprising a plurality of plasmonic nanoparticles for performance of the ELISA;
b. an illumination source arranged in proximity to the digital microfluidic cartridge for providing light;
c. an optical measurement device arranged in proximity to the digital microfluidic cartridge for obtaining optically detectable readings including one or a combination of light intensity, color, and hue; and
d. optionally, a thermal control mechanism for controlling the operating temperature of the digital microfluidic cartridge.

51. The system of claim 50, wherein two or more illumination sources are provided to support multiple sensing elements.

52. The system of claim 50, wherein the illumination source comprises a light source for wavelengths from about 400 nm to about 800 nm.

53. The system of claim 50, wherein the illumination source comprises a light source for any color of light.

54. The system of claim 50, wherein two or more optical measurement devices are provided to support multiple sensing elements.

55. The system of claim 50, wherein the optical measurement device comprises a charge coupled device, a photodetector, a spectrometer, a photodiode array, a camera, a smartphone camera, or any combinations thereof.

56. The system of claim 50, wherein the thermal control mechanism comprises Peltier elements and resistive heaters.

57. The system of claim 50, further comprising a controller for providing processing capabilities, executing software instructions, and controlling the DMF cartridge, the illumination source, the optical measurement device, and the thermal control mechanism.

58. The system of claim 57, wherein the controller is electrically coupled to the DMF cartridge, the illumination source, the optical measurement device, and the thermal control mechanism via a DMF interface.

59. The system of claim 58, wherein the DMF interface comprises a pluggable interface for connecting mechanically and electrically to the DMF cartridge.

60. The system of claim 50, wherein the ELISA comprises:
a. introducing a sample fluid potentially comprising the target analyte to a capture biomolecule within the DMF cartridge, wherein binding of the target analyte to the capture biomolecule forms a target-capture biomolecule complex, and wherein, optionally, one or both of the capture biomolecule and a plasmonic particle is immobilized on a surface;
b. introducing an antibody that binds directly or indirectly to the target analyte at a different site than the capture biomolecule, wherein:
(i) an enzyme is conjugated directly or indirectly to the antibody, or
(ii) the enzyme is conjugated to the capture biomolecule;
c. introducing a substrate for the enzyme;
d. introducing a plasmonic particle;
e. measuring an optically detectable change caused by one or a combination of etching, growth, aggregation, or altered interparticle distance of the plasmonic particle in the vicinity of the target-capture biomolecule complex, wherein the etching, growth, aggregation, or altered interparticle distance is in response to a product or byproduct generated by a reaction between the enzyme and the substrate, wherein the amount of enzyme-substrate reactions is proportional to the number of target analytes bound to the capture biomolecules; and
f. optionally, quantifying an amount of the target analyte present in the sample based on the optically detectable change.

61. The system of claim 60, wherein the target analyte is a protein.

62. The system of claim 60, wherein the plasmonic particles are nanoparticles having one or more particle dimensions less than about 100 nm.

63. The system of claim 62, wherein the plasmonic particles comprise more than one material.

64. The method of claim 63, wherein the particles comprise one or a combination of a dielectric, semiconductor, or polymeric material.

65. The method of claim 64, wherein the semiconductor layer is magnetic, paramagnetic or superparamagnetic.

66. The system of claim 62, wherein the plasmonic particles comprise more than one layer and at least one of the layers comprises a plasmonic layer.

67. The system of claim 66 wherein the plasmonic particles comprise core-shell particles.

68. The system of claim 67 wherein the core-shell particle comprises a silver core or a gold core and an outer porous (or mesoporous) metal oxide layer, thereby facilitating enzyme catalyzed etching of the particle core for optical detection of the target analyte.

69. The method of claim 68, wherein the metal oxide layer comprises silicon dioxide (SiO2@Ag or SiO2@Au).

70. The system of claim 60, wherein the nanoparticles comprise one or a combination of core-shell particles, rattle-type particles, hollow particles, porous particles, bimetallic particles, gold nanorods, gold nanourchins, and particles consisting of a single metal composition.
